(19) United States
(12) Patent Application Publication
(10) Pub. No.: US 2010/0004253 A1
(43) Pub. Date: Jan. 7, 2010

(54) BIOMARKERS OF TARGET MODULATION, EFFICACY, DIAGNOSIS AND/OR PROGNOSIS FOR RAF INHIBITORS

(75) Inventors: Natasha Aziz, Emeryville, CA (US); Edward Moler, Emeryville, CA (US); Darrin Stuart, Emeryville, CA (US); Carla Heise, Emeryville, CA (US); Kim Aardelen, Emeryville, CA (US)

Correspondence Address: NOVARTIS VACCINES AND DIAGNOSTICS INC., INTELLECTUAL PROPERTY- X100B, P.O. BOX 8097, Emeryville, CA, 94662-8097, US

(73) Assignee: Novartis AG

(21) Appl. No.: 12/441,888

(22) PCT Filed: Sep. 19, 2007
(86) PCT No.: PCT/US07/78946

§ 371 Date: May 27, 2009

Related U.S. Application Data
(60) Provisional application No. 60/845,601, filed on Sep. 19, 2006.

(51) Int. Cl. A61K 31/497; C12Q 1/68; A61K 31/4439; A61K 31/47

(52) U.S. Cl. 514/252.1; 435/6; 514/338; 514/314

(57) ABSTRACT

Methods of utilizing biomarkers to identify patients for treatment or to monitor response to treatment are taught herein. Alterations in levels of gene expression of the biomarkers, particularly in response to Raf kinase inhibition, are measured and identifications or adjustments may be made accordingly.

The present invention relates generally to the field of pharmacogenomics and in particular to the use of biomarkers for identifying patients suitable for treatment as well as to methods of following their response to methods of treatment.

An effort to understand an individual patient's response or disease progression is the topic of present day research. Indeed, the field of pharmacogenomics or pharmacogenetics utilizes genomic data, pharmacology, and medicine, and often relies on advanced research tools to correlate genetic variability to one or more of predisposition to a disease and/or its progression, as well as therapeutic response to a drug or therapeutic regimen. Typically, multiple genes are analyzed simultaneously in a large-scale, genome-wide approach.

Proliferative cell disorders such as cancers usually develop through the accumulation of a series of mutations in the patient's DNA within a subpopulation of cells. These mutations may confer a survival advantage on the cells that causes them to grow and spread in an uncontrolled manner that is deleterious to the surrounding tissues. The particular set of mutations may be unique to an individual patient's tumor. Cancers of the same tissue or organ in different individuals may have originated from different sets of mutations, though certain mutations may be prevalent among particular cancer types. The characteristic set of mutations will determine how the cancer cells behave, and in particular, their likelihood of response to a given therapeutic regimen.

One may characterize the genetic alterations in a tumor by using advanced research tools that measure the genetic sequence of the tumor's DNA, or the RNA or proteins that are the expression of the altered DNA. It is a goal of current research to identify characteristics of an individual's tumor that are predictive of the likelihood of that tumor's response to various therapeutic treatments and to identify biomarkers that are modulated in response to treatment, thus improving patient care. Thus, one or more genes would be identified where presence of particular genetic mutations in the DNA, or their levels of expression, either as RNA transcripts or as proteins, or a combination of these factors, would be predictive of the likelihood that a particular treatment would affect the tumor in a manner that would be beneficial to the patient.

One main purpose is to determine which variations in individuals or subpopulations, associated with their genetics or the genetic characteristics of their disease, factor into drug efficacy and to create suitable tests, including diagnostic tests. Drugs that are tailored for patients with a particular genetic sequence, or for diseases characterized by particular genetic alterations, may thus be produced. The tests may also be used to guide treatment decisions, such as which drug or drug combination is most likely to be beneficial to the patient, and what dosing and schedule is most appropriate. Diagnostic tests and genetic profiling will help avoid the expense and the potentially detrimental trial-and-error approach to the suitability of a particular treatment regimen or a particular dosage level.

While the era of customized drugs may be coming, methods that utilize genetic information to identify specific individuals or subgroups for a particular type of treatment or optimization of a treatment may be immediately put to use today.

An individual's response to a particular treatment or predisposition to disease and the correlation to a particular gene of interest has been documented. It is now believed that cancer chemotherapy is limited by the predisposition of specific populations to drug toxicity or poor drug response. For a review of the use of germline polymorphisms in clinical oncology, see Lenz, H. J. (2004) J. Clin. Oncol. 22 (13):2519-2521. For a review of pharmacogenetic and pharmacogenomics in therapeutic antibody development for the treatment of cancer, see Yan and Beckman (2005) Biotechniques 39:565-568.

Results from numerous studies suggest several genes may play a major role in the principal pathways of cancer progression and recurrence, and that the corresponding germ-line polymorphisms may lead to significant differences at transcriptional and/or translational levels. Polymorphism has been linked to cancer susceptibility (oncogenes, tumor suppressor genes, and genes of enzymes involved in metabolic pathways) of individuals. In patients younger than 35 years, several markers for increased cancer risk have been identified. Cytochrome P4501A1 and gluthathione S-transferase MI genotypes influence the risk of developing prostate cancer in younger patients. Similarly, mutations in the tumor suppressor gene, p53, are associated with brain tumors in young adults.

This approach may be extended to mutations that are specific to cancer cells, and not otherwise found in the patient's genome. For instance, it has been demonstrated clinically in patients with gastrointestinal stromal tumors (GIST) treated with the drug Gleevec (imatinib mesylate; Novartis) that particular activating mutations in the genes KIT and PDGFA are linked to higher response rates to the drug, see J Clin Oncol. 2003 Dec. 1; 21 (23):4342-9.

By measuring changes in gene expression of cancer cell lines or an in vivo model of cancer induced by treatment with a particular therapeutic agent, one may characterize the cells' response to that agent. This approach provides insight into the mechanism of the drug, including what biological processes or pathways it impacts. Such information can help guide the treatment of patients, by providing expectations as to which genes will change in response to treatment. An assay of those genes from a sample collected from a patient post-treatment could then be used to determine whether the drug was having the intended effect, and by extension, whether the dose or schedule should be altered, or the regimen discontinued. This approach would improve efficacy by ensuring that patients receive the most appropriate treatment.

By the way of further background, kinases known to be associated with tumorigenesis include the Raf serine/threonine kinases.

The Raf serine/threonine kinases are essential components of the Ras/Mitogen-Activated Protein Kinase (MAPK) signaling module that controls a complex transcriptional program in response to external cellular stimuli. Raf genes code for highly conserved serine-threonine-specific protein kinases which are known to bind to the Ras oncogene. They are part of a signal transduction pathway believed to consist of receptor tyrosine kinases, p21 ras, Raf protein kinases, Mek1 (ERK activator or MAPKK) kinases and ERK (MAPK) kinases, which ultimately phosphorylate transcription factors. In this pathway, Raf kinases are activated by Ras and phosphorylate and activate two isoforms of Mitogen-Activated Protein Kinase (called Mek1 and Mek2), that are dual specificity threonine/tyrosine kinases. Both Mek isoforms activate Mitogen Activated Kinases 1 and 2 (MAPK, also called Extracellular Ligand Regulated Kinase 1 and 2 or Erk1 and Erk2). The MAPKs phosphorylate many substrates including cytosolic proteins and ETS family of transcription factors and in so doing set up their transcriptional program. Raf kinase participation in the Ras/MAPK pathway influences and regulates many cellular functions such as proliferation, differentiation, survival, oncogenic transformation and apoptosis.

Both the essential role and the position of Raf in many signaling pathways have been demonstrated from studies using deregulated and dominant inhibitory Raf mutants in mammalian cells as well as from studies employing biochemical and genetic techniques model organisms. In many cases, the activation of Raf by receptors that stimulate cellular tyrosine phosphorylation is dependent on the activity of Ras, indicating that Ras functions upstream of Raf. Upon activation, Raf-1 then phosphorylates and activates Mek1, resulting in the propagation of the signal to downstream effectors, such as MAPK (Crews et al. (1993) Cell 74:215). The Raf serine/threonine kinases are considered to be the primary Ras effectors involved in the proliferation of animal cells (Avruch et al. (1994) Trends Biochem. Sci. 19:279).

Raf kinase has three distinct isoforms, Raf-1 (c-Raf), A-Raf, and B-Raf, distinguished by their ability to interact with Ras, to activate MAPK kinase pathway, tissue distribution and sub-cellular localization (Marias et al., Biochem. J. 351:289-305, 2000; Weber et al., Oncogene 19:169-176, 2000; Pritchard et al., Mol. Cell. Biol. 15:6430-6442, 1995).

Activating mutation of one of the Ras genes can be seen in about 20% of all tumors and the Ras/Raf/MEK/ERK pathway is activated in about 30% of all tumors (Bos et al., Cancer Res. 49:4682-4689, 1989; Hoshino et al., Oncogene 18:813-822, 1999). Recent studies have shown that B-Raf mutation in the skin nevi is a critical step in the initiation of melanocytic neoplasia (Pollock et al., Nature Genetics 25: 1-2, 2002). Furthermore, most recent studies have disclosed that activating mutation in the kinase domain of B-Raf occurs in about 66% of melanomas, 12% of colon carcinoma and 14% of liver cancer (Davies et al., Nature 417:949-954, 2002) (Yuen et al., Cancer Research 62:6451-6455, 2002) (Brose et al., Cancer Research 62:6997-7000, 2002). It is also present in about 30% of ovarian low-grade serous carcinomas (Shih and Kurman, Am J Pathol, 164:1511-1518, 2004) and 35-70% papillary thyroid carcinomas (Xing et al., J Clin Endocrinology, 90:6373-6379, 2005).

Melanoma, which continues to represent a significant unmet medical need, is a complex multigenic disease with a poor prognosis, especially in the advanced metastatic state. Activating somatic mutations in the B-Raf proto-oncogene have recently been discovered in a variety of malignancies, and most frequently in melanoma. Approximately 70% of melanoma express a mutated and activated form of B-Raf (V600E), making it an excellent target for drug development. Furthermore, another 10-15% of melanomas express mutant N-Ras, further demonstrating the importance of the MAPK pathway in the growth and survival of melanoma cells.

Inhibitors of the Ras/Raf/MEK/ERK pathway at the level of Raf kinases can potentially be effective as therapeutic agents against tumors with over-expressed or mutated receptor tyrosine kinases, activated intracellular tyrosine kinases, tumors with aberrantly expressed Grb2 (an adapter protein that allows stimulation of Ras by the Sos exchange factor) as well as tumors harboring activating mutations of Raf itself. In the early clinical trials, inhibitors of Raf-1 kinase that also inhibit B-Raf have shown promise as therapeutic agents in cancer therapy (Crump, Current Pharmaceutical Design 8:2243-2248, 2002; Sebastien et al., Current Pharmaceutical Design 8: 2249-2253, 2002).

Disruption of Raf expression in cell lines through the application of RNA antisense technology has been shown to suppress both Ras and Raf-mediated tumorigenicity (Kolch et al., Nature 349:416-428, 1991; Monia et al., Nature Medicine 2 (6):668-675, 1996). It has also been shown that the administration of deactivating antibodies against Raf kinase or the co-expression of dominant negative Raf kinase or dominant negative MEK, the substrate of Raf kinase, leads to the reversion of transformed cells to the normal growth phenotype (see Daum et al., Trends Biochem. Sci 1994, 19:474-80; Fridman et al. J. Biol. Chem. 1994, 269:30105-8).

Several Raf kinase inhibitors have been described as exhibiting efficacy in inhibiting tumor cell proliferation in vitro and/or in vivo assays (see, e.g., U.S. Pat. Nos. 6,391,636, 6,358,932, and 6,268,391). Other patents and patent applications suggest the use of Raf kinase inhibitors for treating leukemia (see, e.g., U.S. Pat. Nos. 6,268,391, and published U.S. Patent Application Nos. 20020137774; 20010016194; and 20010006975), or for treating breast cancer (see, e.g., U.S. Pat. Nos. 6,358,932 and 6,268,391, and published U.S. Patent Application No. 20010014679).

It would be particularly beneficial to be able to determine in a patient having a cell proliferative disease whether such disease involves one or more components of the Ras/Raf/MEK/ERK pathway. It would also be beneficial to be able to identify patients with a good likelihood of treatment of a cell proliferative disease with a Raf kinase inhibitor and to monitor the response of those patients.

### SUMMARY OF THE INVENTION

One embodiment of the invention relates to a method of identifying a patient for treatment. The method may optionally include an administration of a Raf kinase inhibitor to the patient. Gene expression is determined from a biological sample from the patient, specifically to detect the presence and/or measure the alteration in level of expression of biomarkers disclosed herein.

Another embodiment of the invention comprises a method of monitoring response of a patient to treatment. The method may include the step of administration of a Raf kinase inhibitor to the patient prior to measurement of gene expression on a biological sample obtained from the patient. Alternatively, monitoring may be conducted on a sample obtained from a patient who has previously been treated so that the administration step by one practicing the method of monitoring response need not performed. The response of the patient is evaluated based the detection of gene expression of at least one biomarker from the tables. Detection and/or alteration in the level of expression of at least one biomarker compared to baseline may be indicative of the response of the patient to the treatment. The pattern of expression level changes may be indicative of a favorable response or of an unfavorable one.

Another aspect of the invention is a method of treating a cell proliferative disorder in a patient. A therapeutically effective amount of an agent that alters gene expression level compared to baseline of at least one of the biomarkers from the tables is administered to the patient for treatment of the disorder. The patient is selected based on evidence of gene expression of at least one of the biomarkers. The agent is preferably CHIR-265 or an agent with an inhibitory profile similar to CHIR-265.

Yet another embodiment of the invention is a method of identifying an agent for treatment of a cell proliferative disorder.

A further embodiment of the invention is a method of identifying a Raf kinase inhibitory agent for treatment or further development of the agent.

Also included are data sets of the biomarkers of any of the tables of biomarkers disclosed herein.

### DETAILED DESCRIPTION OF THE PREFERRED EMBODIMENTS

The invention is an example of translational medicine at work, wherein patients may be treated selectively based on their particular genetic profile.

The biomarkers noted herein may be advantageously utilized to identify patients for treatment and to monitor their response to treatment. For example, dosage amounts may be adjusted, additional therapies may be introduced, toxic response or other adverse events may be foreshadowed and forestalled, or treatment may be discontinued, depending upon the response of the patient to the Raf kinase inhibitor as measured by the expression profile. In addition, the methods taught herein may be utilized to study the mechanism of action of CHIR-265 or other Raf kinase inhibitors on a molecular level. Thereafter, rational combinations for treatment may be based on information learned regarding the molecular mechanism of action.

### DEFINITIONS AND TECHNIQUES

The practice of the present invention will employ, unless otherwise indicated, conventional techniques of immunology, molecular biology, microbiology, cell biology and recombinant DNA, which are within the skill of the art. See e.g., Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL and ANIMAL CELL CULTURE (R. I. Freshney, ed. (1987)).

As used herein, certain terms have the following defined meanings.

As used in the specification and claims, the singular form “a”, “an” and “the” include plural references unless the context clearly dictates otherwise. For example, the term “a cell” includes a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide can comprise modified nucleotides, such as methylated nucleotides and nucleotide analogs. If present, modifications to the nucleotide structure can be imparted before or after assembly of the polymer. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for guanine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

A “gene” refers to a polynucleotide containing at least one open reading frame (ORF) that is capable of encoding a particular polypeptide or protein after being transcribed and translated. A polynucleotide sequence may be used to identify larger fragments or full-length coding sequences of the gene with which they are associated. Methods of isolating larger fragment sequences are known to those of skill in the art.

A “gene product” or alternatively a “gene expression product” refers to the amino acids (e.g., peptide or polypeptide) generated when a gene is transcribed and translated.

The term “polypeptide” is used interchangeably with the term “protein” and in its broadest sense refers to a compound of two or more subunit amino acids, amino acid analogs, or peptidomimetics. The subunits may be linked by peptide bonds. In another embodiment, the subunit may be linked by other bonds, e.g., ester, ether, etc.

As used herein the term “amino acid” refers to either natural and/or unnatural or synthetic amino acids, and both the D and L optical isomers, amino acid analogs, and peptidomimetics. A peptide of three or more amino acids is commonly called an oligopeptide if the peptide chain is short. If the peptide chain is long, the peptide is commonly called a polypeptide or a protein.

The term “isolated” means separated from constituents, cellular and otherwise, in which the polynucleotide, peptide, polypeptide, protein, antibody or fragment(s) thereof, are normally associated within nature. In one aspect of this invention, an isolated polynucleotide is separated from the 3′ and 5′ contiguous nucleotides with which it is normally associated within its native or natural environment, e.g., on the chromosome. As is apparent to those of skill in the art, a non-naturally occurring polynucleotide, peptide, polypeptide, protein, antibody, or fragment(s) thereof, does not require “isolation” to distinguish it from its naturally occurring counterpart. In addition, a “concentrated”, “separated” or “diluted” polynucleotide, peptide, polypeptide, protein, antibody or fragment(s) thereof, is distinguishable from its naturally occurring counterpart in that the concentration or number of molecules per volume is greater in a “concentrated” version or less than in a “separated” version than that of its naturally occurring counterpart. A polynucleotide, peptide, polypeptide, protein, antibody, or fragment(s) thereof, which differs from the naturally occurring counterpart in its primary sequence or, for example, by its glycosylation pattern, need not be present in its isolated form since it is distinguishable from its naturally occurring counterpart by its primary sequence or, alternatively, by another characteristic such as glycosylation pattern. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra.

As used herein, “expression” refers to the process by which polynucleotides are transcribed into mRNA and/or the process by which the transcribed mRNA is subsequently translated into peptides, polypeptides or proteins. If the polynucleotide is derived from genomic DNA, expression may include splicing of the mRNA in a eukaryotic cell. “Differentially expressed” as applied to a gene, refers to the differential production of the mRNA transcribed and/or translated from the gene or the protein product encoded by the gene. A differentially expressed gene may be overexpressed or underexpressed as compared to the expression level of a normal or control cell. However, as used herein overexpression generally is at least 1.25 fold or, alternatively, at least 1.5 fold or, alternatively, at least 2 fold expression, or alternatively, at least 4 fold expression over that detected in a normal or healthy counterpart cell or tissue. The term “differentially expressed” also refers to nucleotide sequences in a cell or tissue which are expressed where silent in a control cell or not expressed where expressed in a control cell.

A high expression level of the gene may occur because of over expression of the gene or an increase in gene copy number. The gene may also be translated into more protein because of deregulation of a negative regulator.

A “gene expression profile” refers to a pattern of expression of a set of genes that recurs in multiple samples and reflects a property shared by those samples, such as tissue type, response to a particular treatment, or activation of a particular biological process or pathway in the cells. Furthermore, a gene expression profile differentiates between samples that share that common property and those that do not with better accuracy than would likely be achieved by assigning the samples to the two groups at random. A gene expression profile may be used to predict whether samples of unknown status share that common property or not. Some variation between the levels of the individual genes of the set and the typical profile is to be expected, but the overall similarity of the expression levels to the typical profile is such that it is statistically unlikely that the similarity would be observed by chance in samples not sharing the common property that the expression profile reflects.

An expression “database” denotes a set of stored data that represent a collection of sequences, which in turn represent a collection of biological reference materials.

The term “cDNAs” refers to complementary DNA, i.e. mRNA molecules present in a cell or organism made into cDNA with an enzyme such as reverse transcriptase. A “cDNA library” is a collection of all of the mRNA molecules present in a cell or organism, all turned into cDNA molecules with the enzyme reverse transcriptase, then inserted into “vectors” (other DNA molecules that can continue to replicate after addition of foreign DNA). Exemplary vectors for libraries include bacteriophage (also known as “phage”), viruses that infect bacteria, for example, lambda phage. The library can then be probed for the specific cDNA (and thus mRNA) of interest.

As used herein, “solid phase support” or “solid support”, used interchangeably, is not limited to a specific type of support. Rather a large number of supports are available and are known to one of ordinary skill in the art. Solid phase supports include silica gels, resins, derivatized plastic films, glass beads, cotton, plastic beads, alumina gels, microarrays, and chips. As used herein, “solid support” also includes synthetic antigen-presenting matrices, cells, and liposomes. A suitable solid phase support may be selected on the basis of desired end use and suitability for various protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel®, Rapp Polymere, Tubingen, Germany), or polydimethylacrylamide resin (obtained from Milligen/Biosearch, California).

A polynucleotide also can be attached to a solid support for use in high throughput screening assays. PCT WO 97/10365, for example, discloses the construction of high density oligonucleotide chips. See also, U.S. Pat. Nos. 5,405,783; 5,412,087; and 5,445,934. Using this method, the probes are synthesized on a derivatized glass surface to form chip arrays. Photoprotected nucleoside phosphoramidites are coupled to the glass surface, selectively deprotected by photolysis through a photolithographic mask and reacted with a second protected nucleoside phosphoramidite. The coupling/deprotection process is repeated until the desired probe is complete.

As an example, transcriptional activity may be assessed by measuring levels of messenger RNA using a gene chip such as the Affymetrix HG-U133-Plus-2 GeneChips. High-throughput, real-time quanititation of RNA (of hundreds of genes simultaneously) thus becomes possible in a reproducible system.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

A polynucleotide or polynucleotide region (or a polypeptide or polypeptide region) has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art, for example those described in CURRENT PROTOCOLS IN MOLECULAR BIOLOGY (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: www.ncbi.nlm.nih.gov/cgi-bin/BLAST.

The term “cell proliferative disorders” shall include dysregulation of normal physiological function characterized by abnormal cell growth and/or division or loss of function. Examples of “cell proliferative disorders” includes but is not limited to hyperplasia, neoplasia, metaplasia, and various autoimmune disorders, e.g., those characterized by the dysregulation of T cell apoptosis.

Hyperplasia is a form of controlled cell proliferation involving an increase in cell number in a tissue or organ, without significant alteration in structure or function. Metaplasia is a form of controlled cell growth in which one type of fully differentiated cell substitutes for another type of differentiated cell. Metaplasia can occur in epithelial or connective tissue cells. Atypical metaplasia involves a somewhat disorderly metaplastic epithelium.

As used herein, the terms “neoplastic cells,” “neoplastic disease,” “neoplasia,” “tumor,” “tumor cells,” “cancer,” and “cancer cells,” (used interchangeably) refer to cells which exhibit relatively autonomous growth, so that they exhibit an aberrant growth phenotype characterized by a significant loss of control of cell proliferation (i.e., de-regulated cell division). Neoplastic cells can be malignant or benign. A metastatic cell or tissue means that the cell can invade and destroy neighboring body structures.

The term “cancer” refers to cancer diseases that can be beneficially treated by the inhibition of Raf kinase, including, for example, solid cancers, such as carcinomas (e.g., of the lungs, pancreas, thyroid, ovaries, bladder, breast, prostate, liver, or colon), melanomas, myeloid disorders (e.g., myeloid leukemia, multiple myeloma and erythroleukemia), and adenomas (e.g., villous colon adenoma) and sarcomas (e.g., osteosarcoma).

“Suppressing” tumor growth indicates a growth state that is curtailed when compared to growth without contact with educated, antigen-specific immune effector cells. Tumor cell growth can be assessed by any means known in the art, including, but not limited to, measuring tumor size, determining whether tumor cells are proliferating using a ³H-thymidine incorporation assay, measuring glucose uptake by FDG-PET (fluorodeoxyglucose positron emission tomography) imaging, or counting tumor cells. “Suppressing” tumor cell growth means any or all of the following states: slowing, delaying and stopping tumor growth, as well as tumor shrinkage.

A “composition” is also intended to encompass a combination of active agent and another carrier, e.g., compound or composition, inert (for example, a detectable agent or label) or active, such as an adjuvant, diluent, binder, stabilizer, buffers, salts, lipophilic solvents, preservative, adjuvant or the like. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term “carrier” further includes a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl-.quadrature.-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers with the additional provisio that they be acceptable for use in vivo. For examples of carriers, stabilizers and adjuvants, see Martin REMINGTON'S PHARM. SCI., 15th Ed. (Mack Publ. Co., Easton (1975) and Williams & Williams, (1995), and in the “PHYSICIAN'S DESK REFERENCE”, 52^{nd} ed., Medical Economics, Montvale, N.J. (1998).

An “effective amount” is an amount sufficient to effect beneficial or desired results. An effective amount can be administered in one or more administrations, applications or dosages.

A “subject,” “individual” or “patient” is used interchangeably herein, which refers to a vertebrate, preferably a mammal, more preferably a human. Mammals include, but are not limited to, murines, simians, humans, farm animals, sport animals, and pets.

Raf kinase has three distinct isoforms, Raf-1 (c-Raf), A-Raf, and B-Raf, distinguished by their ability to interact with Ras, to activate MAPK kinase pathway, tissue distribution and sub-cellular localization (Marias et al., Biochem. J. 351:289-305, 2000; Weber et al., Oncogene 19:169-176, 2000; Pritchard et al., Mol. Cell. Biol. 15:6430-6442, 1995), and by genomic sequence. Raf-1 is identified by the US National Institutes of Health with Entrez-Gene ID 5894 and is mapped to chromosome location 3p25 (http://www.ncbi.nlm.nih.gov/books/bv.fcgi?rid=handbook.chapter.ch19). A-Raf and B-Raf Entrez Gene IDs are 369 and 673 with chromosomal locations Xp11.4 and 7q34, respectively. The first Raf kinase discovered, RAF-1, was identified in a search for oncogenic viral sequences (Rapp et al., Proc. Nat. Acad. Sci. 80:4218-4222, 1983). A-Raf and B-Raf were subsequently cloned and identified as Raf family members ((Mark et al., Proc. Nat. Acad. Sci. 83:6312-6316, 1986 and Sithanandam et al., Oncogene 5:1775-1780, 1990).

An “inhibitor” of Raf or of Raf kinase as used herein binds or blocks or diminishes the effect of the Raf kinase. Examples include, but are not limited to CHIR-265 and related compounds, BAY 43-9006, and siRNA. Other examples include ISIS-5132 (CGP-69846A), ODN-698, and ISIS-13650.

A “Raf inhibitor,” “Raf kinase inhibitor,” or “inhibitor of Raf” useful in conjunction with the invention preferably refers to a compound that exhibits an IC₅₀ with respect to Raf kinase activity of no more than about 100 μM and more typically not more than about 50 μM, as measured in a Raf/Mek Filtration Assay. The Raf/Mek Filtration Assay is as described in WO 03/082272 and reproduced in Example 3. Preferred isoforms of Raf kinase especially useful in conjunction with the present invention include A-Raf, B-Raf, and C-Raf (Raf-1). “IC₅₀” is that concentration of inhibitor which reduces the activity of an enzyme (e.g., Raf kinase) to half-maximal level. Compounds such as CHIR-265 have been discovered to exhibit inhibitory activity against Raf. Compounds useful in conjunction with the methods of the present invention preferably exhibit an IC₅₀ with respect to Raf of no more than about 10 μM, more preferably, no more than about 5 μM, even more preferably not more than about 1 μM, and most preferably, not more than about 200 nM, as measured in Raf kinase assays. A preferred agent, CHIR-265, has shown the characteristics presented below:

| | | | | | C-RAF | B-RAF |
|---|---|---|---|---|---|---|
| | | | | B-RAF | (WILD | (WILD |
| | VEGFR2 | PDGFRβ | CKIT | (V600E) | TYPE) | TYPE) |
| IC₅₀ | 0.07 | 0.003 | 0.02 | 0.019 | 0.005 | 0.07 |
| (μM) | | | | | | |
| EC₅₀ | 0.19 | 0.79 | 1.1 | 0.12 | 0.3-1.0 | 0.3-1.0 |
| (μM) | | | | | | |

As used herein, the phrase “MAPK signal transduction pathway” is an abbreviation that stands for Mitogen activated protein kinase signal transduction pathway in a module that is formed of the Ras-Raf-MEK1-ERK signaling molecules.

A “biomarker” is a distinctive indicator or specific feature or characteristic of a biological process or event. As used herein, a biomarker is a gene. A biomarker may be especially useful for measuring the progress of a disease or the response to a given treatment. In addition to assessing prognosis, in some instances, it may be used to diagnose an illness or screen for patients within a category, such as those most likely to respond to a certain type of treatment. A biomarker may also be useful in guiding the development or administration of an agent for treatment of a disease.

As noted above, the invention provides methods of identifying patients suitable for treatment and methods of monitoring response in patients receiving treatment. Also included within the scope of the invention are methods of treating a cell proliferative disorder. The methods include adjusting dosage amounts, altering inhibitors, or otherwise altering treatment by utilizing the biomarkers disclosed herein.

The present invention also provides a screen for various agents and methods that may supplement or replace the anti-Raf kinase therapy known in the art. In one aspect, the agent, alone or in combination with another agent or therapy method, is provided to the patient. After administration, a sample from the patient is assessed for expression of one or more biomarkers identified herein and then compared to a baseline.

Further details regarding the practice of the invention are discussed below.

### Biomarkers

Panels of genes have now been identified, whose expression correlates with the inhibition of Raf kinase. The presence or absence of gene expression or the level or amount of gene expression of one or more of the biomarkers identified herein may be used to guide treatment decisions and measure responsiveness of the patient to a given type of treatment. For example, detection of the presence or lack thereof of gene expression or alteration of the level of gene expression compared to baseline of one or more of the biomarkers identified in Tables I-XX provides information regarding whether a patient may be a suitable candidate for treatment by CHIR-265 or another Raf kinase inhibitor or drug with similar inhibitory profile.

Changes in gene expression level in response to the Raf kinase inhibitor were tested as presented in the experimental examples and those deemed to be of statistical significance were utilized to generate the tables disclosed herein.

Within the parameters of this experiment, and as discussed in greater detail below, the biomarkers of Table I generally correlate with significant alterations in expression level. The biomarkers of Table II generally correlate with alterations in expression level of 5-fold or greater compared to baseline, and the biomarkers of Table III generally correlate with alterations in expression level of 10-fold or greater compared to baseline, whereas the biomarkers of Table IV generally correlate with alterations in expression level of 30-fold or greater compared to baseline. It should be noted that these fold-change numbers (as well as other numbers used to generate tables according to the invention) are indications of relative changes in expression level as per the experiments reported herein and may not represent absolute numbers. Also, changes in transcription level of any of the genes may correlate with a greater or lesser change at the level of protein. Table V is a subset of Table I showing the biomarkers of Table I that are particularly preferred because their gene products are secreted. Table VI is a subset of Table I showing the biomarkers of Table I that are even more particularly preferred because their gene products are secreted and they showed 10-fold or greater alteration in expression level in this experiment. Table VII is a subset showing the biomarkers of Table I that are preferred according to a different aspect of the invention because they showed significant alterations in expression level compared to baseline sustained over several time points.

Also within the parameters of this experiment and as discussed in greater detail below, the biomarkers of Table VIII generally correlate with significant alterations in expression level. Table VIII was generated by examining the data resulting from the protocol of Example 1 in an alternate way, following the six queries of Example 1's section B to identify the genes showing greatest modulation by the Raf kinase inhibitor as compared to matching control. Table IX is a preferred subset of Table VIII. Tables X and XI are subsets of Table IX that generally correlate with down-regulation and up-regulation, respectively, of the biomarkers listed in Table IX. Tables XII through XIV show various subsets of Tables IX with secreted gene products. Tables XV through XVII show various subsets of Table IX that are likely to have their gene products located on a cell surface.

Also of particular interest are the biomarkers listed on Tables XVIII and XIX, since they are involved in decreased glucose uptake in tumors. These biomarkers may be useful indicators of suppression of tumor growth, as discussed in greater detail in Example 2. Table XX is a most preferred subset of the biomarkers from Tables XVIII and XIX. These biomarkers relate to glucose uptake and/or metabolism and may provide a molecular explanation for why glucose uptake is decreased in the experiment.

Any or all of the biomarkers presented in the tables are of interest and those in the most preferred subsets such as Table IV, V, VI, VII, XII, XV, and XX may especially be of interest.

As noted, of particular interest are biomarkers whose gene product is secreted, thereby allowing the gathering of information and assessment of a patient's condition to be quick and efficient. For example, hepatocyte growth factor (HGF, Entrez Gene ID 3082) is secreted in serum and is believed indicative of cell proliferation, invasiveness, and angiogenesis, among others. Serum HGF levels have been reported to be reflective of aggressive disease in patients with invasive breast cancer (Sheen-Chen, et al., “Serum Levels of Hepatocyte Growth Factor in Patients with Breast Cancer,” Cancer Epidemiol. Biomarkers Prev., 14 (3):715-7 (2005)) and non-small cell lung cancer (Siegfried, et al., “The Clinical Significance of Hepatocyte Growth Factor for Non-Small Cell Lung Cancer,” Ann. Thorac. Surg., 66:1915-8 (1998)). Thus, measurement of the HGF biomarker, particularly as a secreted gene product, is an easy and inexpensive tool available to health care providers in prognosis and determining appropriateness of certain courses of treatment.

As is apparent to one of skill in the art, gene expression can be measured by detecting the presence or absence, or presence and/or absolute or relative quantity of a gene expression product (e.g., RNA, mRNA, or the protein or polypeptide transcript) or the alteration in gene copy number. In some embodiments, altered expression is likely the result of an increase in copy number. In alternative embodiment, altered expression is likely the result of the loss of function of another gene such as a tumor suppressor or other negative regulator. In yet a further embodiment, expression is altered by the “turning on” of an enhancer. Accordingly, the specific method used to detect altered expression, as compared to the control or baseline, may be different and dependent on the particular biomarker selected. In yet further embodiments, the method requires analysis of gene expression of one or more predetermined biomarkers by more than one method, e.g., by use of immunohistochemical and molecular techniques such as a gene chip or array.

### Tables

Tables I through XX are presented below and constitute an integral part of this disclosure. In each of Tables I through XX, the biomarkers are shown with Entrez Gene ID Number (referring to the National Cancer Institute database identifier), Gene Symbol, and Gene Description.

Table I is a list of biomarkers whose alteration of level of gene expression compared to baseline is indicative of activity related to Raf kinase inhibition.

Table II is a preferred subset of Table I according to one aspect of the invention, listing biomarkers generally having a higher level of alteration of gene expression compared to baseline in response to Raf kinase inhibition.

Table III is more preferred subset of Table I according to one aspect of the invention, listing biomarkers generally having an even higher level, in comparison to Table II, of alteration of gene expression compared to baseline in response to Raf kinase inhibition.

Table IV is an even more preferred subset of Table I according to one aspect of the invention listing biomarkers generally having the highest level of alteration of gene expression compared to baseline in response to Raf kinase inhibition.

Table V is a preferred subset of Table I according to a second aspect of the invention, listing biomarkers whose gene product is secreted.

Table VI is a preferred subset of Table I according to a third aspect of the invention, listing biomarkers generally exhibiting a high level of alteration compared to baseline and whose gene product is secreted.

Table VII is a preferred subset of Table I according to a fourth aspect of the invention, listing biomarkers generally exhibiting a measurably sustained high level of alteration compared to baseline.

Table VIII is a list of biomarkers according to yet another aspect of the invention whose alteration of level of gene expression compared to baseline is indicative of activity related to Raf kinase inhibition. Table VIII lists the 7345 biomarkers whose gene expression level was deemed significantly altered using an alternate analysis method than that used to generate Tables I through VII.

Table IX is a preferred subset of Table VIII and lists biomarkers with most significant alteration levels at each step of the analysis.

Table X is a subset of Table IX showing that portion of biomarkers identified in Table IX that are preferably down-regulated.

Table XI is a subset of Table IX showing that portion of biomarkers identified in Table IX that are preferably up-regulated.

Table XII is a preferred subset of Table IX according to another aspect of the invention, listing biomarkers whose gene product is likely to be secreted.

Table XIII is a subset of Table XII, showing that portion of biomarkers identified in Table XII that are preferably down-regulated.

Table XIV is a subset of Table XII, showing that portion of biomarkers identified in Table XII that are preferably up-regulated.

Table XV is a preferred subset of Table IX according to another aspect of the invention, listing biomarkers whose gene product is likely to be located on a cell surface.

Table XVI is a subset of Table XV, showing that portion of biomarkers dentified in Table XV that are preferably down-regulated.

Table XVII is a subset of Table XV, showing that portion of biomarkers identified in Table XV that are preferably up-regulated.

Table XVIII is a preferred list of biomarkers according to yet another aspect of the invention. Table XVIII lists biomarkers that are transporters and/or glycolysis pathway members and whose down-regulation compared to baseline is indicative of activity related to Raf kinase inhibition and tumor regression.

Table XIX is a preferred list of biomarkers according to yet another aspect of the invention. Table XIX lists biomarkers that are transporters and/or glycolysis pathway members and whose up-regulation compared to baseline is indicative of activity related to Raf kinase inhibition and tumor regression.

Table XX is a preferred list of biomarkers taken from Tables XVIII and XIX.

Lengthy table referenced here
US20100004253A1-20100107-T00001
Please refer to the end of the specification for access instructions.

The gene expression of the biomarkers of Table I or any of its subsets, Tables II through VII, may be down- or up-regulated in response to the inhibition of Raf kinase. The same is true of the biomarkers of Table VIII. Similarly, gene expression of Table IX, Table XII, or Table XV biomarkers may be down- or up-regulated in response to the inhibition of Raf kinase, although it is preferred that such biomarkers are down- or up-regulated in accordance with the guidance provided by Tables X/XI, Tables XIII/XIV, or Tables XVI/XVII, respectively. In some instances, the detection of the presence of gene expression of one of the biomarkers may be sufficient to identify the patient for treatment or provide indication of a favorable response to treatment. In other instances, one may prefer the guidance provided by a higher level of alteration of gene expression. This is typically within the judgment of the treatment provider.

Further, in some instances, one may find identifying the most suitable patients for treatment for a particular cell proliferative disorder or following the response of those patients may best be accomplished by detecting an alteration in level of gene expression of two or more biomarkers or by a specific combination of biomarkers or even direction of alteration of gene expression. For example, a particular two of the biomarkers identified in Table I may be most correlated with a given condition and, thus, guide a certain treatment. Alternatively, a ratio of the relative levels of gene expression of two particular biomarkers may be indicative of the suitability of a given treatment for a patient. It is also contemplated that a particular condition may have a signature such as the up-regulation of one or more particular biomarker or biomarkers and/or the down-regulation of one or more other particular biomarker or biomarkers. The biomarkers may be over-expressed or under-expressed in a sample obtained from the patient relative to baseline.

In the methods of the invention, determining presence of gene expression of a biomarker encompasses detecting presence or absence of gene expression of the biomarker, as well as determining an alteration in the level of gene expression of the biomarker, such as by measuring gene expression level of the biomarker and comparing the measured gene expression level to baseline. Selecting a patient evidencing gene expression of a biomarker includes evidencing as by detecting the presence of gene expression of the biomarker. As used herein, determining presence of gene expression may be direct or indirect, such as by obtaining results from a sample or evaluating results obtained from a sample by another. In a method of monitoring response of a patient to treatment, evaluating response of a patient based on detection of the presence of gene expression of a biomarker includes, for example, continuing treatment if gene expression of at least one biomarker is detected, as well as discontinuing or altering treatment if such gene expression is not detected.

The alteration in the level of gene expression may be compared to a baseline level. A baseline level may be established in several ways. For example, in a method of monitoring response of a patient to treatment, a biological sample may be obtained from the patient and tested for measurement of gene expression prior to introduction of a Raf kinase inhibitor to the patient. Thus, the profile of gene expression levels, if any, of biomarkers in a treatment-naïve individual may serve as a baseline for that individual and later tests performed on samples obtained once treatment has begun may be compared to the individual's baseline. Alternatively, a baseline may be established through creation of a guide that consolidates information on gene expression levels taken from a pool of healthy or treatment-naïve individuals or even from an appropriate cell culture. Further, information on baseline levels of gene expression of particular biomarkers may be gathered from published sources or a gene database. Thus, baseline could be obtained or established from gene expression level information of similar patient populations not known to have been treated with a Raf kinase inhibitor. Baseline could also be represented by the gene expression level of a reference standard, such as a reference sample (or average of several reference samples) obtained from lung, pancreas, thyroid, or other tissue of the patient or another. If the reference sample is not obtained from the patient, then it is preferably of the same tissue type as the biological sample obtained from the patient.

In one aspect, a biological sample is obtained from the patient after receipt of an amount of inhibitor of Raf kinase, whether a therapeutically effective amount or a sub-therapeutically effective amount, which may be adequate for some purposes. The sample is preferably isolated from impurities, or may otherwise be a functional derivative of the starting material obtained from the patient or another. Cell or tissue samples used for this invention encompass body fluid (including but not limited to blood, serum, or plasma), solid tissue samples, tissue cultures or cells derived therefrom and the progeny thereof, and sections or smears prepared from any of these sources, or any other samples that may contain genetic information. The biological sample may be obtained, for example, from lung, pancreas, thyroid, ovary, bladder, breast, prostate, liver, colon, myeloid tissue, skin, or tumor tissue. The sample may be obtained from a region showing evidence a cell proliferative disorder in the patient. Measurement of the expression of the biomarkers is described in further detail below.

### Inhibitors of Raf

Compounds which are inhibitors of Raf, and particularly of the enzyme Raf kinase, are useful in conjunction with the methods of the invention. Since the enzyme is a downstream effector of p21 Ras, Raf inhibitors are useful in pharmaceutical compositions for human or veterinary use where inhibition of the Raf kinase pathway is indicated, e.g., in the treatment of tumors and/or cancerous cell growth mediated by Raf kinase. In particular, such compounds are useful in the treatment of human or animal, e.g., murine cancer, since the progression of these cancers is dependent upon the Ras protein signal transduction cascade and therefore is susceptible to treatment by interruption of the cascade by inhibiting Raf kinase activity. Such compounds are useful in treating solid cancers, such as, for example, carcinomas (e.g., of the lungs, pancreas, thyroid, ovaries, bladder, breast, prostate, liver, or colon), melanomas, myeloid disorders (e.g., myeloid leukemia, multiple myeloma and erythroleukemia), or adenomas (e.g., villous colon adenoma) or sarcomas (e.g., osteosarcoma).

Some examples of inhibitors of Raf kinase include the following compounds: CHIR-265 (Chiron Corporation); BAY 43-9006 (sorafenib)(Nexavar® Bayer AG); ISIS-5132 (CGP-69846A)(Isis Pharmaceuticals); ODN-698 (Novartis); ISIS-13650 (Isis Pharmaceuticals); LE-AON, LEraf-AON, LE-AON c-Raf, LE-5132 (NeoPharm); N-[3-[6-(3-Oxo-1,3-dihydro-2-benzofuran-5-ylamino)pyrazin-2-yl]phenyl]acetamide (Cancer Research UK); PLX-4720, PLX-3204, PLX-3331, PLX-4718, PLX-4735, PLX-4032 (Plexxikon); N-[5-[3-(1-Cyano-1-methylethyl)benzamido]-2-methylphenyl]-4-oxo-3,4-dihydroquinazoline-6-carboxamide (AstraZeneca); and N-[5-[3-(1-Cyano-1-methylethyl)benzamido]-2-methylphenyl]-3-methyl-4-oxo-3,4-dihydroquinazoline-6-carboxamide (AstraZeneca). CHIR-265 has the following structures (drawn in the two alternative tautomeric forms):

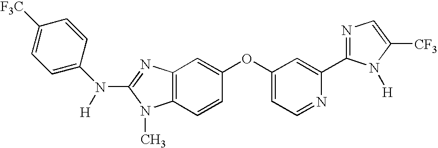

### {1-Methyl-5-[2-(5-trifluoromethyl-1H-imidazol-2-yl)-pyridin-4-yloxy]-1H benzoimidazol-2-yl}-(4-trifluoromethyl-phenyl)-amine and

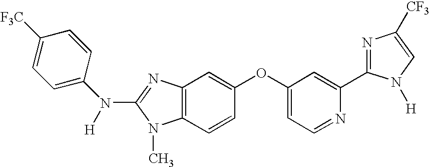

### {1-Methyl-5-[2-(4-trifluoromethyl-1H-imidazol-2-yl)-pyridin-4-yloxy]-1H-benzoimidazol-2-yl}-(4-trifluoromethyl-phenyl)-amine

As is art recognized, tautomeric forms of a compound merely represent alternative structures that can be in equilibrium depending upon, for example, the site of protonation of the imidazole amino group. Both structures, in fact, are intended to represent CHIR-265.

CHIR-265 is an exemplary Raf-inhibitory compound for use in the methods of this invention, CHIR-265 exhibits potent inhibition of the MAPK signaling pathway. The compound is a potent inhibitor of B-Raf, c-Raf, mutant B-Raf, and mutant Ras in biochemical assays, demonstrating inhibition of mutant B-Raf activity (IC₅₀ of 0.019 μM), inhibition of B-Raf activity (IC₅₀ of 0.068 μM), and inhibition of c-Raf activity (IC₅₀ of 0.005 μM). Treatment with the compound caused tumor regression in three mutant B-Raf xenograft models (A375M, MEXF276, and HT29) tested, and tumor growth inhibition in a K-Ras (HCT116) driven xenograft model. CHIR-265 also showed inhibition of the Raf pathway in other mutant B-Raf cell lines tested, including SKMEL28, A2058, G361, COLO205, SKMEL31, and MALME-3M, as well as a K-Ras cell line (SW620) and an N-Ras cell line (SKMEL2). Furthermore, assays in preclinical models indicated that CHIR-265 showed a dose and time dependent inhibition of both MEK target phosphorylation and the signaling molecules downstream from Raf in the MAPK pathway including BIM, Cyclin D1, p27Kip and pAKT.

This compound and related compounds have been described in several patents and applications, the entire disclosures of which are incorporated herein by reference and for all purposes: U.S. Ser. No. 60/712,539, filed Aug. 30, 2005, U.S. Ser. No. 60/731,591, filed Oct. 27, 2005, U.S. Ser. No. 60/774,684, filed Feb. 17, 2006, U.S. Ser. No. 60/713,108, filed Aug. 30, 2005, U.S. Ser. No. 11/513,959, filed Aug. 30, 2006, and U.S. Ser. No. 11/513,745, filed Aug. 30, 2006.

Each of the references listed above is hereby incorporated by reference in its entirety and for all purposes as if fully set forth herein.

### Measurement of Gene Expression

As noted previously, the measurement of gene expression is performed on a sample, preferably a biological sample, obtained from the patient. For example, the patient may undergo a blood draw or tissue biopsy and the measurement may be made on the resulting sample. Depending upon the technique utilized, the test may be performed on an isolated fraction of the sample or in situ.

Detection of the presence of gene expression of the biomarker of interest and/or detection of the level of alteration in the gene expression compared to baseline may be made utilizing standard techniques.

Detection can be by any appropriate method, including for example, detecting the quantity of mRNA transcribed from the gene or the quantity of cDNA produced from the reverse transcription of the mRNA transcribed from the gene or the quantity of the polypeptide or protein encoded by the gene. These methods can be performed on a sample by sample basis or modified for high throughput analysis. Additionally, databases containing quantitative full or partial transcripts or protein sequences isolated from a cell sample can be searched and analyzed for the presence and amount of transcript or expressed gene product.

In assaying for an alteration in mRNA level, nucleic acid contained in the aforementioned samples is first extracted according to standard methods in the art. For instance, mRNA can be isolated using various lytic enzymes or chemical solutions according to the procedures set forth in Sambrook et al. (1989), supra or extracted by nucleic-acid-binding resins following the accompanying instructions provided by manufacturers. The mRNA of the biomarker contained in the extracted nucleic acid sample is then detected by hybridization (e.g. Northern blot analysis) and/or amplification procedures according to methods widely known in the art or based on the methods exemplified herein.

Nucleic acid molecules having at least 10 nucleotides and exhibiting sequence complementarity or homology to the biomarkers described herein find utility as hybridization probes. It is known in the art that a “perfectly matched” probe is not needed for a specific hybridization. Minor changes in probe sequence achieved by substitution, deletion, or insertion of a small number of bases do not affect the hybridization specificity. In general, as much as 20% base-pair mismatch (when optimally aligned) can be tolerated.

In certain embodiments, it will be advantageous to employ probes or primers in combination with an appropriate means, such as a label, for detecting hybridization and therefore complementary sequences. A wide variety of appropriate indicator means are known in the art, including fluorescent, radioactive, enzymatic, or other ligands, such as avidin/biotin, which are capable of giving a detectable signal. In preferred embodiments, one will likely desire to employ a fluorescent label or an enzyme tag, such as urease, alkaline phosphatase, or peroxidase, instead of radioactive or other environmental undesirable reagents. In the case of enzyme tags, calorimetric indicator substrates are known, which can be employed to provide a means visible to the human eye or spectrophotometrically, to identify specific hybridization with complementary nucleic acid-containing samples.

Hybridization reactions can be performed under conditions of different “stringency.” Relevant conditions include temperature, ionic strength, time of incubation, the presence of additional solutes in the reaction mixture such as formamide, and the washing procedure. Higher stringency conditions are those conditions, such as higher temperature and lower sodium ion concentration, which require higher minimum complementarity between hybridizing elements for a stable hybridization complex to form. Conditions that increase the stringency of a hybridization reaction are widely known and published in the art. See, for example, (Sambrook, et al., (1989), supra).

Briefly, multiple RNAs are isolated from cell or tissue samples as described above. Optionally, the gene transcripts can be converted to cDNA. A sampling of the biomarker transcript(s) is/are subjected to sequence-specific analysis and quantified. These gene transcript sequence abundances are compared to the baseline.

Alternatively any one of gene copy number, transcription, or translation of a biomarker can be determined using known techniques. For example, an amplification method such as PCR may be useful. General procedures for PCR are taught in MacPherson et al., PCR: A PRACTICAL APPROACH, (IRL Press at Oxford University Press (1991)). However, PCR conditions used for each application reaction are empirically determined. A number of parameters influence the success of a reaction. Among them are annealing temperature and time, extension time, Mg²⁺ ATP concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination.

In one aspect, the biomarkers are detected and quantitated by hybridization to a probe that specifically hybridizes to the appropriate probe for that biomarker. The probes also can be attached to a solid support for use in high throughput screening assays using methods known in the art. PCT WO 97/10365 and U.S. Pat. Nos. 5,405,783, 5,412,087 and 5,445,934, for example, disclose the construction of high density oligonucleotide chips which can contain one or more of the sequences disclosed herein. Using the methods disclosed in U.S. Pat. Nos. 5,405,783, 5,412,087 and 5,445,934, the probes of this invention are synthesized on a derivatized glass surface. Photoprotected nucleoside phosphoramidites are coupled to the glass surface, selectively deprotected by photolysis through a photolithographic mask, and reacted with a second protected nucleoside phosphoramidite. The coupling/deprotection process is repeated until the desired probe is complete.

In one aspect, the expression level of the biomarker is determined through exposure of a nucleic acid sample to the probe-modified chip. Extracted nucleic acid is labeled, for example, with a fluorescent tag, preferably during an amplification step. Hybridization of the labeled sample is performed at an appropriate stringency level. The degree of probe-nucleic acid hybridization is quantitatively measured using a detection device, such as a confocal microscope. See U.S. Pat. Nos. 5,578,832 and 5,631,734.

In an alternative embodiment, the method is performed by the detecting and comparing of two or more biomarkers that have been pre-determined to be predictive of a therapeutic response. In a yet further embodiment, a plurality of biomarkers, e.g., see Tables I through XX, supra, are used in the method of this invention. In these embodiments, the biomarkers or probes that specifically hybridize and recognize the biomarker of interest are arranged on a high density oligonucleotide probe array that provides an effective means of monitoring expression of a multiplicity of genes.

In another preferred embodiment, the methods of this invention are used to monitor expression of the genes which specifically hybridize to the probes of this invention in response to defined stimuli, such as a drug or biologic.

In one embodiment, the hybridized nucleic acids are detected by detecting one or more labels attached to the sample nucleic acids. The labels may be incorporated by any of a number of means well known to those of skill in the art. However, in one aspect, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g. fluorescein-labeled UTP and/or CTP) incorporates a label in to the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or ³²P) enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

As described in more detail in WO 97/10365, the label may be added to the target (sample) nucleic acid(s) prior to, or after the hybridization. These are detectable labels that are directly attached to or incorporated into the target (sample) nucleic acid prior to hybridization. In contrast, “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see LABORATORY TECHNIQUES IN BIOCHEMISTRY AND MOLECULAR BIOLOGY, Vol. 24: Hybridization with Nucleic Acid Probes, P. Tijssen, ed. Elsevier, N.Y. (1993).

The nucleic acid sample also may be modified prior to hybridization to the high density probe array in order to reduce sample complexity thereby decreasing background signal and improving sensitivity of the measurement using the methods disclosed in WO 97/10365.

Results from the chip assay are typically analyzed using a computer software program. See, for example, EP 0717 113 A2 and WO 95/20681. The hybridization data is read into the program, which calculates the expression level of the targeted gene(s). The figures may be compared against existing data sets of gene expression levels for diseased and healthy individuals. A correlation between the obtained data and that of a set of a predetermined baseline identifies patients likely to be responsive to the therapy.

Also within the scope of this application is a database useful for the identification of patients likely to respond to a predetermined therapy, e.g., anti-Raf kinase therapy, wherein the database contains a combination of base line gene expression data against which the patient sample can be compared using bioinformatic techniques known in the art.

The pre-determined baseline information is stored in a digital storage medium such that a data processing system for standardized representation of the genes that identify patients that are responsive to therapy. The data processing system is useful to analyze gene expression between two samples. A suitable sample is isolated from the patient and then the genotype or phenotype of the cell or sample is determined using methods known in the art. In one aspect, the nucleic acids of the biomarkers if present in the sample are sequenced and transcribed to code. The sequences (in code form) from the sample are compared with the sequence(s) present in the database using homology search techniques. Greater than 90%, or alternatively, greater than 95% or alternatively, greater than or equal to 97% sequence identity between the test sequence and at least one sequence identified by the biomarkers identified in Tables I through XX is a positive indication that the polynucleotide from a biomarker has been isolated from the patient sample.

Expression level of the biomarker can also be determined by examining the protein product. Determining the protein level involves (a) providing a biological sample containing expression product of the biomarker; and (b) measuring the amount of any immunospecific binding that occurs between an antibody that selectively recognizes and binds to the expression product of the biomarker in the sample, in which the amount of immunospecific binding indicates the level of the biomarker expression, or (c) monitoring the binding of a protein that positively or negatively regulates a biomarker. This information is then compared to a pre-determined base line and analyzed to identify those patients suitable for therapy.

A variety of techniques are available in the art for protein analysis. They include but are not limited to radioimmunoassays, ELISA (enzyme linked immunosorbent assays), “sandwich” immunoassays, immunoradiometric assays, in situ immunoassays (using e.g., colloidal gold, enzyme or radioisotope labels), western blot analysis, immunoprecipitation assays, immunofluorescent assays, flow cytometry, immunohistochemistry, confocal microscopy, enzymatic assays, and PAGE-SDS.

Antibodies that specifically recognize and bind to the protein products of the expression products of the biomarkers are required for immunoassays. These may be purchased from commercial vendors or generated and screened using methods well known in the art. See Harlow and Lane (1988) supra. and Sambrook et al. (1989) supra.

### Treatment

Inhibition of Raf kinase is a useful avenue for treatment of cellular proliferative disease and particularly neoplastic disease. A patient may be beneficially treated by administration of an inhibitor of Raf kinase, particularly a small molecule inhibitor (SMI) of Raf kinase. Thus, treatment according to the invention may constitute administration of one or more small molecule inhibitors of Raf kinase, such as those disclosed herein.

Some disease models in which the genetic profiling methods taught herein are especially useful include melanoma, thyroid cancer, ovarian cancer, colon cancer, liver cancer, pancreatic cancer, and lung cancer.

Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents may be empirically adjusted.

More particularly, an agent administered according to the invention may be administered for therapy by any suitable route including oral, rectal, nasal, topical (including transdermal, aerosol, buccal, and sublingual), vaginal, parenteral (including subcutaneous, intramuscular, intravenous and intradermal) and pulmonary. It will also be appreciated that the preferred route will vary with the condition and age of the recipient and the disease being treated.

Ideally, the agent should be administered to achieve peak concentrations of the active compound at sites of disease. This may be achieved, for example, by the intravenous injection of the agent, optionally in saline or orally administered, for example, as a tablet, capsule, or syrup containing the active ingredient. Desirable blood levels of the agent may be maintained by a continuous infusion to provide a therapeutic amount of the active ingredient within disease tissue. In a specific embodiment, it may be desirable to administer pharmaceutical compositions locally to the area in need of treatment; this may be achieved by, for example, and not by way of limitation, local infusion during surgery, by injection, or by means of a catheter. The use of operative combinations may provide therapeutic combinations requiring a lower total dosage of each component agent than may be required when each individual therapeutic compound or drug is used alone, thereby reducing adverse effects.

While it is possible for the agent to be administered alone, it is preferable to present it as a pharmaceutical formulation comprising at least one active ingredient together with one or more pharmaceutically acceptable carriers, and optionally other therapeutic agents. Each carrier must be “acceptable” in the sense of being compatible with the other ingredients of the formulation and not injurious to the patient.

Pharmaceutical compositions utilized according to the methods of the invention may take the form of tablets, lozenges, granules, capsules, pills, ampoules, suppositories, or aerosol form. They may also take the form of suspensions, solutions, or emulsions of the active ingredient in aqueous or nonaqueous diluents, syrups, granulates, or powders. In addition to the key active ingredients, the pharmaceutical compositions can also contain other pharmaceutically active compounds or a plurality of compositions of the invention.

Formulations include those suitable for oral, rectal, nasal, topical (including transdermal, buccal and sublingual), vaginal, parenteral (including subcutaneous, intramuscular, intravenous and intradermal) and pulmonary administration. The formulations may conveniently be presented in unit dosage form and may be prepared by any methods well known in the art of pharmacy. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both and then, if necessary, shaping the product.

Formulations suitable for oral administration may be presented as discrete units such as capsules, cachets or tablets, each containing a predetermined amount of the active ingredient; as a powder or granules; as a solution or suspension in an aqueous or non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin or sucrose and acacia; and mouthwashes comprising the active ingredient in a suitable liquid carrier.

Pharmaceutical compositions for topical administration according to the present invention may be formulated as an ointment, cream, suspension, lotion, powder, solution, paste, gel, spray, aerosol, or oil. Alternatively, a formulation may comprise a patch or a dressing such as a bandage or adhesive plaster impregnated with active ingredients and optionally one or more excipients or diluents.

If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the agent through the skin or other affected areas. Examples of such dermal penetration enhancers include dimethylsulfoxide and related analogues.

The oily phase of the emulsions of a composition used according to this invention may be constituted from known ingredients in a known manner. While this phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulphate.

The choice of suitable oils or fats for the formulation is based on achieving the desired cosmetic properties, since the solubility of the active compound in most oils likely to be used in pharmaceutical emulsion formulations is very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with a suitable base comprising, for example, cocoa butter or a salicylate.

Formulations suitable for vaginal administration may be presented as pessaries, tampons, creams, gels, pastes, foams, or spray formulations containing in addition to the agent, such carriers as are known in the art to be appropriate.

Formulations suitable for nasal administration, wherein the carrier is a solid, include a coarse powder having a particle size, for example, in the range of about 20 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the agent.

Formulations suitable for parenteral administration include aqueous and non-aqueous isotonic sterile injection solutions that may contain anti-oxidants, buffers, bacteriostats, and solutes that render the formulation isotonic with the blood of the intended recipient. Other suitable formulations include aqueous and non-aqueous sterile suspensions that may include suspending agents, thickening agents and liposomes or other microparticulate systems that are designed to target the compound to blood components or one or more organs. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Various delivery systems are known and can be used to administer a therapeutic agent in accordance with the methods of the invention, e.g., encapsulation in liposomes, microparticles, microcapsules, expression by recombinant cells, receptor-mediated endocytosis (See e.g., Wu and Wu (1987) J. Biol. Chem. 262:4429-4432), construction of a therapeutic nucleic acid as part of a retroviral or other vector, etc. Methods of delivery include but are not limited to intra-arterial, intra-muscular, intravenous, intranasal and oral routes.

As noted, the compounds can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq. (1976).

Preferred unit dosage formulations are those containing a daily dose or unit, daily subdose, as herein above-recited, or an appropriate fraction thereof, of an agent. Effective amounts of the compounds generally include any amount sufficient to detectably inhibit Raf activity by Raf kinase activity assays known to those having ordinary skill in the art or by detecting an inhibition or alleviation of symptoms of cancer.

The amount of active ingredient that may be combined with the carrier materials to produce a single dosage form will vary depending upon the subject treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease for which the patient is undergoing therapy. The therapeutically effective amount for a given situation can be readily determined by routine experimentation and is within the skill and judgment of the ordinary clinician.

For purposes of the present invention, a therapeutically effective dose will generally be a total daily dose administered to a host in single or divided doses and may be in amounts, for example, of from 0.001 to 1000 mg/kg body weight daily and more preferred from 1.0 to 30 mg/kg body weight daily. Dosage unit compositions may contain such amounts of submultiples thereof to make up the daily dose.

Therapeutic agents utilized according to this invention, include, but are not limited to small molecules. They may be polynucleotides, peptides, antibodies, antigen presenting cells and include immune effector cells that specifically recognize and act upon cells expressing the gene of interest. One can determine if a subject or patient will be beneficially treated by the use of agents by screening one or more of the agents against tumor cells isolated from the subject or patient using methods known in the art.

Alternatively, small molecule inhibitors may be used in combination with other treatments. For instance, inhibitors that are not small molecules, e.g. biologicals, polynucleotides, gene therapy, etc. may be used in conjunction in a combination protocol for treatment. In some cases, the small molecule Raf kinase inhibitor may be used primarily as an initial aid in identifying patient candidates and other types of inhibitors may be used subsequently, or vice versa. In another alternative, one inhibitor may be used prior to a gene expression level measurement step and another may be used subsequently.

As noted, while the Raf kinase inhibitory compounds can be administered as the sole active pharmaceutical agent, they can also be used in combination with one or more other agents used in the treatment of cancer. The Raf kinase inhibitory compounds are also useful in combination with known therapeutic agents and anti-cancer agents, and combinations of the presently disclosed compounds with other anti-cancer or chemotherapeutic agents are within the scope of the invention. Examples of such agents can be found in Cancer Principles and Practice of Oncology, V. T. Devita and S. Hellman (editors), 6^{th} edition (Feb. 15, 2001), Lippincott Williams & Wilkins Publishers. The health care provider should be able to discern which combinations of agents would be useful based on the particular characteristics of the drugs and the cancer involved.

Methods for treating Raf kinase related disorders in a human or animal subject in need of such treatment may include administering to the subject an amount of a Raf kinase inhibitory compound effective to reduce or prevent tumor growth in the subject in combination with at least one additional agent for the treatment of cancer. A number of suitable anticancer agents to be used as combination therapeutics are contemplated for use in conjunction with the methods of the present invention. Advantageous administration of numerous anticancer agents such as: agents that induce apoptosis; polynucleotides (e.g., ribozymes); polypeptides (e.g., enzymes); drugs; biological mimetics; alkaloids; alkylating agents; antitumor antibiotics; antimetabolites; hormones; platinum compounds; monoclonal antibodies conjugated with anticancer drugs, toxins, and/or radionuclides; biological response modifiers (e.g. interferons [e.g. IFN-alpha, etc.] and interleukins [e.g. IL-2, etc.], etc.); adoptive immunotherapy agents; hematopoietic growth factors; agents that induce tumor cell differentiation (e.g. all-trans-retinoic acid, etc.); gene therapy reagents; antisense therapy reagents and nucleotides; tumor vaccines; inhibitors of angiogenesis, and the like in a combination therapy may be beneficial.

In preferred embodiments, anticancer agents to be used in combination with Raf inhibitory compounds comprise agents that induce or stimulate apoptosis. Agents that induce apoptosis include, but are not limited to, radiation (e.g., W); kinase inhibitors (e.g., Epidermal Growth Factor Receptor [EGFR] kinase inhibitor, Vascular Growth Factor Receptor [VGFR] kinase inhibitor, Fibroblast Growth Factor Receptor [FGFR] kinase inhibitor, Platelet-derived Growth Factor Receptor [PGFR] I kinase inhibitor, and Bcr-Abl kinase inhibitors such as STI-571, Gleevec, and Glivec]); antisense molecules; antibodies [e.g., Herceptin and Rituxan]; anti-estrogens [e.g., raloxifene and tamoxifen]; anti-androgens [e.g., flutamide, bicalutamide, finasteride, aminoglutethamide, ketoconazole, and corticosteroids]; cyclooxygenase 2 (COX-2) inhibitors [e.g., Celecoxib, meloxicam, NS-398, and non-steroidal antiinflammatory drugs (NSAIDs)]; and cancer chemotherapeutic drugs [e.g., irinotecan (Camptosar), CPT-11, fludarabine (Fludara), dacarbazine (DTIC), dexamethasone, mitoxantrone, Mylotarg, VP-16, cisplatinum, 5-FU, Doxrubicin, Taxotere or taxol]; cellular signaling molecules; ceramides and cytokines; and staurosprine, and the like.

Further anti-cancer agents for use in combination include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. A Raf kinase inhibitory compound is also useful when co-administered with radiation therapy.

Estrogen receptor modulators are compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethyl-propanoate, 4,4′-dihydroxybenzophenone-2,4-dinitrophenyl-hydrazone, and SH646.

Androgen receptor modulators are compounds which interfere with or inhibit the binding of androgens to an androgen receptor. Representative examples of androgen receptor modulators include finasteride and other 5α-reductase inhibitors, nilutamide, flutamide, bicalutamide, liarozole, and abiraterone acetate. Retinoid receptor modulators are compounds which interfere or inhibit the binding of retinoids to a retinoid receptor. Examples of retinoid receptor modulators include bexarotene, tretinoin, 13-cis-retinoic acid, 9-cis-retinoic acid, α-difluoromethylornithine, LX23-7553, trans-N-(4′-hydroxyphenyl) retinamide, and N4-carboxyphenyl retinamide.

Cytotoxic and/or cytostatic agents are compounds which cause cell death or inhibit cell proliferation primarily by interfering directly with the cell's functioning or inhibit or interfere with cell mytosis, including alkylating agents, tumor necrosis factors, intercalators, hypoxia activatable compounds, microtubule inhibitors/microtubule-stabilizing agents, inhibitors of mitotic kinesins, inhibitors of kinases involved in mitotic progression, antimetabolites; biological response modifiers; hormonal/anti-hormonal therapeutic agents, haematopoietic growth factors, monoclonal antibody targeted therapeutic agents, topoisomerase inhibitors, proteasome inhibitors and ubiquitin ligase inhibitors. Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX 100, (trans, trans, trans)-bis-mu-(hexane-1,6-diamine)-mu-[diamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, and 4-demethoxy-3-deamino-3-aziridinyl-4-methylsulphonyl-daunorubicin (see WO 00/50032). A representative example of a hypoxia-activatable compound is tirapazamine. Proteasome inhibitors include, but are not limited to, lactacystin and bortezomib. Examples of microtubule inhibitors/microtubule-stabilizing agents include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro4-methoxyphenyl)benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797. Representative examples of topoisomerase inhibitors include topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroOxy-3,5-dimethoxyphenyl]-5,5a,6,8,8a,9-hexahydrofuro(3′,4′:6,7)naphtho(2,3-d)-1,3-dioxol-6-one, 2,3-(methylene-dioxy)-5-methyl-7-hydroxy-8-methoxybenzo[c]-phenanthridinium, 6,9-bis[(2-amino-ethyl)amino]benzo[g]isoquinoline-5,10-dione, 5-(3-aminopropylamino)-7,10-dihydroxy-2-(2-hydroxyethylaminomethyl)-6H-pyrazolo[4,5,1′-de]acridin-6-one, N-[1-[2(diethylamino)-ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna. Examples of inhibitors of mitotic kinesins, such as the human mitotic kinesin KSP, are described in PCT Publications WO 01/30768 and WO 01/98278, WO 03/050,064 (Jun. 19, 2003), WO 03/050,122 (Jun. 19, 2003), WO 03/049,527 (Jun. 19, 2003), WO 03/049,679 (Jun. 19, 2003), WO 03/049,678 (Jun. 19, 2003) and WO 03/39460 (May 15, 2003) and pending PCT Appl. Nos. US03/06403 (filed Mar. 4, 2003), US03/15861 (filed May 19, 2003), US03/15810 (filed May 19, 2003), US03/18482 (filed Jun. 12, 2003) and US03/18694 (filed Jun. 12, 2003). In an embodiment inhibitors of mitotic kinesins include, but are not limited to inhibitors of KSP, inhibitors of MKLP1, inhibitors of CENP-E, inhibitors of MCAK, inhibitors of Kif14, inhibitors of Mphosph1 and inhibitors of Rab6-KIFL.

Inhibitors of kinases involved in mitotic progression include, but are not limited to, inhibitors of aurora kinase, inhibitors of Polo-like kinases (PLK) (e.g., inhibitors of PLK-1), inhibitors of bub-1 and inhibitors of bub-R1. Antiproliferative agents include antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,1-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine and 3-aminopyridine-2-carboxaldehyde thiosemicarbazone. Examples of monoclonal antibody targeted therapeutic agents include those therapeutic agents which have cytotoxic agents or radioisotopes attached to a cancer cell specific or target cell specific monoclonal antibody. Examples include, for example, Bexxar. HMG-CoA reductase inhibitors are inhibitors of 3-hydroxy-3-methylglutaryl-CoA reductase. Compounds which have inhibitory activity for HMG-CoA reductase can be readily identified by using assays well-known in the art such as those described or cited in U.S. Pat. No. 4,231,938 and WO 84/02131. Examples of HMG-CoA reductase inhibitors that may be used include, but are not limited to, lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896) and atorvastatin (LIPITOR®; see U.S. Pat. Nos. 5,273,995, 4,681,893, 5,489,691 and 5,342,952). The structural formulas of these and additional HMG-CoA reductase inhibitors that may be used in conjunction with the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. In an embodiment, the HMG-CoA reductase inhibitor is selected from lovastatin and simvastatin.

Prenyl-protein transferase inhibitors are compounds which inhibit any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase). Examples of prenyl-protein transferase inhibiting compounds include (±)-6-[amino(4-chlorophenyl)(1-methyl-1H-imidazol-5-yl)methyl]-4-(3-chlorophenyl)-1-methyl-2(1H)-quinolinone, (−)-6-[amino(4-chlorophenyl)(1-methyl-1H-imidazol-5-yl)methyl]-4-(3-chlorophenyl)-1-methyl-2(1H)-quinolinone, (+)-6-[amino(4-chlorophenyl)(1-methyl-1H-imidazol-5-yl)methyl]-4-(3-chlorophenyl)-1-methyl-2(1H)-quinolinone, 5(S)-n-butyl-1-(2,3-dimethylphenyl)-4-[1-(4-cyanobenzyl)-5-imidazolylmethyl-2-piperazinone, (S)-1-(3-chlorophenyl)-4-[1-(4-cyanobenzyl)-5-imidazolylmethyl]-5-[2-(ethanesulfonyl)methyl)-2-piperazinone, 5(S)-n-butyl-1-(2-methylphenyl)-4-[1-(4-cyanobenzyl)-5-imidazolylmethyl]-2-piperazinone, 1-(3-chlorophenyl)-4-[1-(4-cyanobenzyl)-2-methyl-5-imidazolylmethyl]-2-piperazinone, 1-(2,2-diphenylethyl)-3-[N-(1-(4-cyanobenzyl)-1H-imidazol-5-ylethyl)carbamoyl]piperidine, 4-{-[4-hydroxymethyl-4-(4-chloropyridin-2-ylmethyl)-piperidine-1-ylmethyl]-2-methylimidazol-1-ylmethyl}benzonitrile, 4-{-5-[4-hydroxymethyl-4-(3-chlorobenzyl)-piperidine-1-ylmethyl]-2-methylimidazol-1-ylmethyl}-benzonitrile, 4-{3-[4-(2-oxo-2H-pyridin-1-yl)benzyl]-3H-imidazol-4-ylmethyl}benzonitrile, 4-{3-[4-(5-chloro-2-oxo-2H-[1,2′]bipyridin-5′-ylmethyl]-3H-imidazol-4-yl-methyl}benzonitrile, 4-{3-[4-(2-oxo-2H-[1,2′]bipyridin-5′-ylmethyl]-3H-imidazol-4-yl-methyl}benzonitrile, 4-[3-(2-oxo-1-phenyl-1,2-dihydropyridin-4-ylmethyl)-3H-imidazol-4-ylmethyl}benzonitrile, 18,19-dihydro-19-oxo-5H,17H-6, 10:12,16-dimetheno-1H-imidazo[4,3-c][1,11,4]dioxaazacyclo-nonadecine-9-carbonitrile, (±)-19,20-dihydro-19-oxo-5H-18,21-ethano-12,14-etheno-6,10-metheno-22H-benzo[d]imidazo[4,3-k]-[1,6,9,12]oxatriaza-cyclooctadecine-9-carbonitrile, 19,20-dihydro-19-oxo-5H,17H-18,21-ethano-6,10:12,16-dimetheno-22H-imidazo[3,4-h][1,8,11,14]oxatriazacycloeicosine-9-carbonitrile, and (.+-.)-19,20-dihydro-3-methyl-19-oxo-5H-18,21-ethano-12,14-etheno-6,10-metheno-22H-benzo[d]imidazo[4,3-k][1,6,9,12]oxa-triazacyclooctadecine-9-carbonitrile. Other examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see European J. of Cancer 35 (9):1394-1401 (1999).

Angiogenesis inhibitors refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-alpha., interleukin-12, pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib (PNAS 89:7384 (1992); JNCI 69:475 (1982); Arch. Opthalmol. 108:573 (1990); Anat. Rec., (238):68 (1994); FEBS Letters 372:83 (1995); Clin, Orthop. 313:76 (1995); J. Mol. Endocrinol. 16:107 (1996); Jpn. J. Pharmacol. 75:105 (1997); Cancer Res. 57:1625 (1997); Cell 93:705 (1998); Intl. J. Mol. Med. 2:715 (1998); J. Biol. Chem. 274:9116 (1999)), steroidal anti-inflammatories (such as corticosteroids, mineralocorticoids, dexamethasone, prednisone, prednisolone, methylpred, betamethasone), carboxyamidotriazole, combretastatin A4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, 17:963-968 (October 1999); Kim et al., Nature, 362:841-844 (1993); WO 00/44777; and WO 00/61186). Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in PCT Publication WO 03/013,526 and U.S. Ser. No. 60/349,925 (filed Jan. 18, 2002). Also contemplated are combinations of small molecule Raf inhibitory compounds with NSAIDs which are selective COX-2 inhibitors (generally defined as those which possess a specificity for inhibiting COX-2 over COX-1 of at least 100 fold as measured by the ratio of IC₅₀ for COX-2 over IC₅₀ for COX-1 evaluated by cell or microsomal assays). Such compounds include, but are not limited to those disclosed in U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, U.S. Pat. No. 5,710,140, issued Jan. 20, 1998, WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999, all of which are hereby incorporated by reference. Representative inhibitors of COX-2 that are useful in the methods of the present invention include 3-phenyl-4-(4-(methylsulfonyl)phenyl)-2-(5H)-furanone; and 5-chloro-3-(4-methylsulfonyl)phenyl-2-(2-methyl-5-pyridinyl)pyridine. Compounds which are described as specific inhibitors of COX-2 and are therefore useful in conjunction with the present invention can be found in the following patents, pending applications and publications, which are herein incorporated by reference: WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, U.S. Pat. No. 5,932,598, issued Aug. 3, 1999, U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, and U.S. Pat. No. 5,710,140, issued Jan. 20, 1998. Other examples of angiogenesis inhibitors include, but are not limited to, endostatin, ukrain, ranpirnase, IM862, 5-methoxy4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RP14610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

Agents that interfere with cell cycle checkpoints are compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

Inhibitors of cell proliferation and survival signaling pathway are pharmaceutical agents that inhibit cell surface receptors and signal transduction cascades downstream of those surface receptors. Such agents include inhibitors of inhibitors of EGFR (for example gefitinib and erlotinib), inhibitors of ERB-2 (for example trastuzumab), inhibitors of IGFR, inhibitors of cytokine receptors, inhibitors of MET, inhibitors of PI3K (for example LY294002), serine/threonine kinases (including but not limited to inhibitors of Akt such as described in WO 02/083064, WO 02/083139, WO 02/083140 and WO 02/083138), inhibitors of Raf kinase (for example BAY-43-9006), inhibitors of MEK (for example CI-1040 and PD-098059) and inhibitors of mTOR (for example Wyeth CCI-779). Such agents include small molecule inhibitor compounds and antibody antagonists.

Apoptosis inducing agents include activators of TNF receptor family members (including the TRAIL receptors).

In certain presently preferred embodiments, representative agents useful in combination with the small molecule Raf kinase inhibitory compounds for the treatment of cancer include, for example, irinotecan, topotecan, gemcitabine, 5-fluorouracil, leucovorin carboplatin, cisplatin, taxanes, tezacitabine, cyclophosphamide, vinca alkaloids, imatinib (Gleevec), anthracyclines, rituximab, trastuzumab, as well as other cancer chemotherapeutic agents.

The above compounds to be employed in combination with the Raf kinase inhibitory compounds such as CHIR-265 will be used in therapeutic amounts as indicated in the Physicians' Desk Reference (PDR) 60th Edition (2006), which is incorporated herein by reference, or such therapeutically useful amounts as would be known to one of ordinary skill in the art.

The Raf kinase inhibitory compounds and the other anticancer agents may be administered at the recommended maximum clinical dosage or at lower doses, within the judgment of the treating physician. Dosage levels of the active compounds in the compositions of the invention may be varied so as to obtain a desired therapeutic response depending on the route of administration, severity of the disease, and the response of the patient. The combination can be administered as separate compositions or as a single dosage form containing both agents. When administered as a combination, the therapeutic agents can be formulated as separate compositions, which are given at the same time or different times, or the therapeutic agents, can be given as a single composition.

Antiestrogens, such as tamoxifen, inhibit breast cancer growth through induction of cell cycle arrest, that requires the action of the cell cycle inhibitor p27Kip. Recently, it has been shown that activation of the Ras-Raf-MAP Kinase pathway alters the phosphorylation status of p27Kip such that its inhibitory activity in arresting the cell cycle is attenuated, thereby contributing to antiestrogen resistance (Donovan et al., J. Biol. Chem. 276:40888, 2001). As reported by Donovan et al., inhibition of MAPK signaling through treatment with MEK inhibitor changed the phosphorylation status of p27 in hormone refractory breast cancer cell lines and in so doing restored hormone sensitivity. Accordingly, in one aspect, Raf kinase inhibitory compounds may be used in the treatment of hormone-dependent cancers, such as breast and prostate cancers, to reverse hormone resistance commonly seen in these cancers with conventional anticancer agents.

In hematological cancers, such as chronic myelogenous leukemia (CML), chromosomal translocation is responsible for the constitutively activated BCR-AB1 tyrosine kinase. The afflicted patients are responsive to Gleevec, a small molecule tyrosine kinase inhibitor, as a result of inhibition of Ab1 kinase activity. However, many patients with advanced stage disease respond to Gleevec initially, but then relapse later due to resistance-conferring mutations in the Ab1 kinase domain. In vitro studies have demonstrated that BCR-Av1 employs the Raf kinase pathway to elicit its effects. In addition, inhibiting more than one kinase in the same pathway provides additional protection against resistance-conferring mutations. Accordingly, Raf kinase inhibitory compounds may be used in combination with at least one additional agent, such as Gleevec, in the treatment of hematological cancers, such as chronic myelogenous leukemia (CML), to reverse or prevent resistance to the at least one additional agent.

Raf kinase inhibitors are useful for treating patients with a need for such inhibitors (e.g., those suffering from cancer mediated by abnormal MAPK signaling). Cancer types mediated by abnormal MAPK signaling include, for example, melanoma, papillary cancer, thyroid cancer, ovarian cancer, colon cancer, pancreatic cancer, non-small cell lung cancer (NSCLC), acute lymphoblastic leukemia (ALL), and acute myeloid leukemia. Abnormal MAPK signaling may be inhibited by administering a compound that inhibits wild-type or mutant forms of Ras, Raf, MEK, or ERK.

The therapeutic compounds in accordance with this aspect of the invention are useful for treating patients with a need for such inhibitors (e.g., those suffering from cancer mediated by abnormal tyrosine kinase receptor signaling). Cancers mediated by abnormal tyrosine kinase receptor signaling include, for example, melanoma, breast cancer, bladder cancer, lung cancer, thyroid cancer, prostate cancer, ovarian cancer, mast cell leukemia, germ cell tumors, small-cell lung carcinoma, gastrointestinal stromal tumors, acute myelogenous leukemia (AML), neuroblastoma, and pancreatic cancer.

### Screens

The invention also includes methods of identifying agents useful for treatment of a cell proliferative disorder or useful to guide a decision to progress agents to further development, as by contacting a candidate agent with a cell line or tissue associated with the disorder and then testing a portion of the cell culture or tissue to measure gene expression level of the biomarkers disclosed herein. For example, the method may include contacting a candidate agent with A375M cells or cells from an A375M-cell-initiated xenograft tumor. As noted earlier, gene expression level may be measured via detecting RNA transcription of at least one biomarker, detecting DNA produced from reverse transcription of RNA transcribed by at least one biomarker, or detecting a polypeptide or protein encoded by at least one biomarker. Preferably the agent is a Raf kinase inhibitor. The methods are useful not only in identifying agents, but also in elucidating the mechanism of action, for example by a candidate agent triggering a signaling pathway. As noted earlier, the biomarkers may be operably linked to a gene chip and presented in a computer readable format for rapid screening.

### EXPERIMENTAL EXAMPLES

### Example 1

Transcriptional activity was assessed by measuring levels of messenger RNA (mRNA) in cells derived from xenograft tumors in mice from an A375M melanoma cancer cell line using Affymetrix HG-U133-Plus-2 GeneChips.

Xenograft tumors were grown in forty nude mice, each 6-8 weeks old, using the A375M melanoma cancer cell line by implanting with 2.5×10⁶ A375M cells subcutaneously in the right flank. When tumor volume reached approximately 200 mm³, the mice were randomized into their respective groups and treatment begun. The A375M melanoma cancer cell line is a B-Raf-mutant driven melanoma cell line.

Animals were dosed orally with 100 mg/kg of CHIR-265 or with vehicle only (no CHIR-265) every other day. The treatment period lasted a total of 28 days. Tumors from vehicle-treated and CHIR-265-treated mice were harvested at the following time points with five animals per timepoint: 8 hours, 24 hours, 192 hours (48 hours after the fourth dose), and 336 hours (48 hours after the seventh dose). Tumors were also harvested from five untreated mice on the first day of the study to serve as naïve controls.

Tumors were harvested in an RNAse treated work area with RNAse treated instruments. Once excised, tumors were placed in RNAse free 15 mL falcon tubes in approximately 5 mL of RNAlater® for four hours at room temperature and then refrigerated overnight at 4° C. Excess RNAlater® was decanted the following day and the tumors stored at −80° C. until RNA was isolated.

RNA was prepared for microarray profiling using the Affymetrix GeneChip ArrayStation running the manufacturer's recommended protocols (http://www.affymetrix.com/products/instruments/specific/arraystation.affx). The target products were hybridized to Affymetrix U133-Plus-2 whole genome microarrays and scanned with an Affymetrix GeneChip Scanner 3000. Bioinformatics analysis was performed on the raw data to provide the results. The manufacturer's recommended quality control criteria were examined during the laboratory processing and data analysis to ensure high-quality results. One sample was determined to fail the quality control criteria, resulting in four Day 14 CHIR-265-treated samples. All other time points had data for five treated and five control samples.

A. Differential expression was determined between the CHIR-265 treated xenografts and the matching vehicle-only controls. An average expression level was determined for each probeset at each time-point and treatment condition by computing the geomean expression level across replicate tumors. For purposes of this experiment, probesets were identified as significantly differential if the ratio of CHIR-265 treated/vehicle-only control was greater than 3-fold up or down with a p-value less than 0.001 in at least one time point. The p-value was determined by a two-tailed t-test assuming unequal variances (Satterwaithe's approximation). The probesets were mapped to genes by blasting the target probe sequences, supplied by Affymetrix, against GenBank.

Of the genes probed on the microarray, 490 were determined to be significantly differential. These biomarkers are listed in Table I.

Any of the genes identified can be used individually or in combination to diagnostically or prognostically determine response of human tumors to Raf kinase inhibitors. Within this set of genes, several interesting subsets can be defined.

Genes with relatively large differentials are particularly promising as biomarker candidates. Of the 490 biomarkers of Table I, 165 of them presented in Table II, have a fold-change of 5 or greater over baseline in at least one time point, and thus Table II represents a preferred subset according to one aspect of the invention. As enumerated in Table III, 45 genes of the 490 have a fold-change of 10 or greater over baseline, and thus represent an even more preferred subset according to one aspect of the invention. Table IV lists 4 genes of the 490 that have a fold-change of 30 or greater over baseline; these biomarkers represent the most preferred subset according to one aspect of the invention.

Genes whose products are secreted can potentially be detected in other tissues, particularly peripheral blood, but possibly in skin, saliva, urine, and other easily accessible tissues. Of the 490 genes, the 77 listed in Table V are identified as secreted and therefore represent an especially useful subset of the biomarkers identified herein according to a second aspect of the invention.

Genes whose products are both secreted and have relatively large fold-changes are especially good candidates as biomarkers, as the samples are easy to collect from subjects and detection results tend to be significant. Of the 490 genes with significant differentials, 17 have a fold-change of greater than 10 and are secreted. These 17 secreted and significantly differential biomarkers are presented in Table VI.

Table VII lists the 189 genes of Table I that exhibited a significant level of alteration compared to baseline over 3 or more consecutive timepoints of the experiment.

One gene in this list, HGF (gene id 3082) is particularly interesting because it has been reported to be detectable in the peripheral blood of breast cancer patients (Sheen-Chen, et. al. “Serum Levels of Hepatocyte Growth Factor in patients with Breast Cancer.” Cancer Epidemiol Biomarkers Prev 14 (2005): 715-717), and also appears relevant to non-small cell lung cancer (Siegfried, et al., “The Clinical Significance of Hepatocyte Growth Factor for Non-Small Cell Lung Cancer,” Ann. Thorac. Surg., 66:1915-8 (1998)).

B. The data obtained from the experiment was also analyzed in an alternate way. Six queries were performed on the data to identify the genes showing greatest modulation by CHIR-265 as compared to the matching vehicle-only controls.

The six queries were as follows:

(1) (Average Day 1, Hour 8 CHIR-265-treated samples)/(Average Day 1 Hour 8 vehicle-treated samples);

(2) (Average Day 1 Hour 24 CHIR-265-treated samples)/(Average Day 1 Hour 24 vehicle-treated samples);

(3) (Average Day 8 CHIR-265-treated samples)/(Average Day 8 vehicle-treated samples);

(4) (75^{th} Percentile Day 14 CHIR-265-treated samples)/(75^{th} percentile Day 14 vehicle-treated samples);

(5) (Average Day 14 CHIR-265-treated samples)/(Average Day 1 Hour 8 CHIR-265-treated samples); and

(6) (95^{th} Percentile all CHIR-265-treated samples)/(95^{th} Percentile all vehicle-treated samples).

Initially, all genes having a p<0.005 in at least one of the six queries was used to generate Table VIII.

Subsequently, for each query, the top 200 (genes upregulated by CHIR-265) and bottom 200 (genes downregulated by CHIR-265) probesets were identified, for a total of 2400 genes.

From the six queries, a subset of 783 probesets, representing 609 unique genes, was selected using the following criteria:

(a) gene-by-gene examination for good margin of differential expression between CHIR-265-treated and vehicle-treated samples, and

(b) all probesets for which the p-value (Student's t-test, 2 tails, equal variance) for all 6 queries p<0.005.

Thus, according to the alternate analysis method, 609 of the genes probed on the microarray were determined to be significantly differential. These biomarkers are listed in Table IX and represent a preferred subset of biomarkers from Table VIII.

As noted earlier, any of the genes identified can be used individually or in combination to diagnostically or prognostically determine response of human tumors to Raf inhibitors. A particular pattern of combinations of biomarkers may be found in biological samples upon exposure to Raf kinase inhibitors even where the tables do not overlap.

Within Table IX, two subsets, Tables X and XI are of particular interest. Table X lists that portion of Table IX whose biomarkers were downregulated by CHIR-265 according to the experimental protocol described and Table XI lists that portion of Table IX whose biomarkers were upregulated by CHIR-265 according to the experimental protocol described. According to one aspect of the invention, a biomarker listed on Table X is preferably modulated in a down-regulated fashion and a biomarker listed on Table XI is preferably modulated in an up-regulated fashion.

In a manner analogous to Tables I and V, another especially useful and therefore preferred subset of Table IX is a set of biomarkers whose gene products are secreted, since these may be easily detected in biological samples such as peripheral blood, skin, saliva, urine, or other easily accessible tissues obtained from the patient. Table IX biomarkers are generally structurally predicted to be secreted due to the presence of a signal sequence and absence of a transmembrane domain. Thus, Table XII represents a preferred portion the of the Table IX biomarkers according to another aspect of the invention, wherein the Table IX biomarkers identified as secreted are listed.

Within Table XII, subsets represented by Table XIII and Table XIV are of particular interest. Table XIII lists that portion of Table XII whose biomarkers were down-regulated by CHIR-265 according to the experimental protocol described and Table XIV lists that portion of Table XII whose biomarkers were upregulated by CHIR-265 according to the experimental protocol described. According to an aspect of the invention, a biomarker listed on Table XIII is preferably modulated in a down-regulated fashion and a biomarker listed on Table XIV is preferably modulated in an up-regulated fashion.

Table XV is a preferred subset of Table IX according to another aspect of the invention. The biomarkers listed in Table XV are those whose gene product is likely to be located on a cell surface. Biomarkers structurally predicted to be on the cell surface were generally selected due to the presence of a transmembrane domain and possibly also a signal sequence. These represent another useful subset of Table IX biomarkers, for example in the situation where tumor cells having proteins indicative of such biomarkers are circulating in the bloodstream of the patient. Such biomarkers may be readily detected from a blood sample via flow cytometry, for example. Immunohistochemistry and Western blots may also be of use in detection of such biomarkers.

Tables XVI and XVII are subsets of Table XV. Table XVI lists biomarkers that were downregulated by CHIR-265 according to the experimental protocol described and Table XVII lists that portion of Table XV whose biomarkers were upregulated by CHIR-265 according to the experimental protocol described. According to an aspect of the invention, a biomarker listed on Table XVI is preferably modulated in a down-regulated fashion and a biomarker listed on Table XVII is preferably modulated in an up-regulated fashion.

### Example 2

As a further means of identifying useful biomarkers, another analysis was completed. The measurement of glucose uptake, as by FDG-PET imaging, has previously been shown to be a useful indicator of suppression of tumor growth. Jallal, B., Keystone Symposium, January 2006; J. Nucl. Med. 2006 June; 47 (6):1059-66. The experiment was set up as in Example 1.

To provide a molecular explanation for decreased glucose uptake in tumors treated with CHIR-265 or another Raf kinase inhibitor, 67 probesets representing solute carrier family members, aquaporins, and genes involved in glucose metabolism (identified through literature searches) and whose expression was modulated by CHIR-265 (p<0.0005 in at least one of queries (1) through (6) as enumerated in Example 1) were identified.

These 67 genes are sorted into those downregulated or upregulated by CHIR-265 and listed in Tables XVIII or XIX, respectively. Thus, biomarkers listed in Table XVIII are preferably down-regulated in response to a Raf kinase inhibitor and biomarkers listed in Table XIX are preferably up-regulated in response to a Raf kinase inhibitor. Table XX is a listing of the most preferred biomarkers (either down-regulated or up-regulated) taken from Tables XVIII and XIX. Table XX lists 19 genes known to be involved in glucose transport or metabolism. Tables XVIII, XIX, and XX are subsets of Table VIII and represent preferred lists of biomarkers according to another aspect of the invention.

### Example 3

### Raf/Mek Filtration Assay

### Buffers

Assay buffer: 50 mM Tris, pH 7.5, 15 mM MgCl₂, 0.1 mM EDTA, 1 mM DTT

Wash buffer: 25 mM Hepes, pH 7.4, 50 mM sodium pyrophosphate, 500 mM NaCl

Stop reagent: 30 mM EDTA

### Materials

Raf, active: Upstate Biotech #14-352

Mek, inactive: Upstate Biotech #14-205

³³P-ATP: NEN Perkin Elmer #NEG 602 h

96 well assay plates: Falcon U-bottom polypropylene plates #35-1190

Filter apparatus: Millipore #MAVM 096 OR

96 well filtration plates: Millipore Immobilon 1 #MAIP NOB

Scintillation fluid: Wallac OptiPhase “SuperMix” #1200-439

### Assay Conditions

Raf approximately 120 pM

Mek approximately 60 nM

³³P-ATP 100 nM

Reaction time 45-60 minutes at room temperature

### Assay Protocol

Raf and Mek were combined at 2× final concentrations in assay buffer (50 mM Tris, pH 7.5, 15 mM MgCl₂. 0.1 mM EDTA and 1 mM DTT) and dispensed 15 μl per well in polypropylene assay plates (Falcon U-bottom polypropylene 96 well assay plates #35-1190. Background levels are determined in wells containing Mek and DMSO without Raf.

To the Raf/Mek containing wells was added 3 μl of 10× of a raf kinase inhibitor test compound diluted in 100% DMSO. The raf kinase activity reaction was started by the addition of 12 μl per well of 2.5×³³P-ATP diluted in assay buffer. After 45-60 minutes, the reactions were stopped with the addition of 70 μl of stop reagent (30 mM EDTA). Filtration plates were pre-wetted for 5 min with 70% ethanol, and then rinsed by filtration with wash buffer. Samples (90 μl) from the reaction wells were then transferred to the filtration plates. The filtration plates were washed 6× with wash buffer using Millipore filtration apparatus. The plates were dried and 100 μl per well of scintillation fluid (Wallac OptiPhase “SuperMix” #1200-439) was added. The CPM is then determined using a Wallac Microbeta 1450 reader.

### Specific Embodiments

The invention includes, but is not limited to, specific embodiments as set forth below:

1. A method of identifying a patient for treatment with a Raf kinase inhibitor, the method comprising:

determining presence of gene expression of at least one biomarker selected from Table I or Table VIII in a biological sample obtained from the patient,

identifying the patient for treatment if the presence of gene expression of the at least one biomarker is detected.

2. The method of 1, wherein the determining step further comprises measuring gene expression level of the at least one biomarker and comparing the measured gene expression level to baseline.
3. The method of 2, wherein baseline is obtained from gene expression level information of similar patient populations not known to have been treated with a Raf kinase inhibitor.
4. The method of 3, wherein the gene expression level information is represented in a gene database.
5. The method of 2, wherein baseline is the gene expression level of a reference standard.
6. The method of 5, wherein the reference standard is not obtained from the patient.
7. The method of 1, wherein the biological sample is obtained from lung, pancreas, thyroid, ovary, bladder, breast, prostate, liver, colon, myeloid tissue, skin, or tumor tissue.
8. The method of 5, wherein the reference standard is a reference sample of the same tissue type as the biological sample obtained from the patient.
9. The method of 1, wherein the biological sample is from a region showing evidence of a cell proliferative disorder.
10. The method of 2, wherein the biomarker is over-expressed in the biological sample relative to baseline.
11. The method of 2, wherein the biomarker is under-expressed in the biological sample relative to baseline.
12. The method of 1 or 2 further comprising the step of administering a Raf kinase inhibitor to the patient.
13. The method of 12, wherein the inhibitor is administered before the biological sample is obtained from the patient.
14. The method of 1 or 2, wherein the determining step further comprises determining gene expression of at least one biomarker selected from a subset of Table I represented by any of Tables II through VII.
15. The method of 1 or 2, wherein the determining step further comprises determining gene expression of at least one biomarker selected from a subset of Table VIII represented by any of Tables IX through XX.
16. The method of 1 or 2, wherein the determining step further comprises determining gene expression of at least two biomarkers selected from Table I and/or Table VIII.
17. The method of 1 or 2, wherein the determining step comprises detecting RNA transcribed by the at least one biomarker.
18. The method of 1 or 2, wherein the determining step comprises detecting DNA produced from reverse transcription of an RNA transcribed by the at least one biomarker.
19. The method of 1 or 2, wherein the determining step comprises detecting a polypeptide or protein encoded by the at least one biomarker.
20. The method of 1 or 2, wherein the at least one biomarker is operably linked to a gene chip.
21. The method of 1 or 2, wherein the at least one biomarker is represented in computer readable format.
22. The method of 1 or 2, wherein the determining step comprises contacting the biological sample with a gene chip comprising any of the biomarkers of Table I and/or Table VIII.
23. The method of 1, wherein the inhibitor is selected from the group consisting of CHIR-265; BAY 43-9006; ISIS-5132; CGP-69846A; ODN-698; ISIS-13650; LE-AON; LEraf-AON; LE-AON c-Raf; LE-5132; N-[3-[6-(3-Oxo-1,3-dihydro-2-benzofuran-5-ylamino)pyrazin-2-yl]phenyl]acetamide; PLX-4720; PLX-3204; PLX-3331; PLX-4718; PLX-4735; PLX-4032; N-[5-[3-(1-Cyano-1-methylethyl)benzamido]-2-methylphenyl]-4-oxo-3,4-dihydroquinazoline-6-carboxamide; and N-[5-[3-(1-Cyano-1-methylethyl)benzamido]-2-methylphenyl]-3-methyl-4-oxo-3,4-dihydroquinazoline-6-carboxamide.
24. The method of 23, wherein the inhibitor is CHIR-265.
25. The method of 1, wherein the biomarker is HGF.
26. The method of 1, wherein the biomarker is not HGF.
27. A method of monitoring response of a patient to treatment with a Raf kinase inhibitor, the method comprising:

determining presence of gene expression of at least one biomarker selected from Table I or Table VIII in a biological sample obtained from a patient who has been administered a Raf kinase inhibitor,

evaluating response of the patient based on detection of the presence of gene expression of the at least one biomarker.

28. The method of 27, wherein the inhibitor has been administered in a therapeutically effective amount.
29. The method of 27 further comprising the step of administering an amount of a Raf kinase inhibitor to the patient.
30. The method of 29, wherein the inhibitor is administered before the biological sample is obtained from the patient.
31. The method of 29, wherein the inhibitor is administered after the evaluating step.
32. The method of 27 or 30 a further comprising obtaining a biological sample from the patient subsequent to the administration of the Raf kinase inhibitor.
33. The method of 27 further comprising altering the treatment based on the evaluating step.
34. The method of 27, wherein detection of the presence of gene expression of the at least one biomarker is indicative of a favorable response to treatment.
35. The method of 27, wherein the determining step further comprises measuring gene expression level of the at least one biomarker and comparing the measured gene expression level to baseline.
36. The method of 35, wherein baseline is obtained from gene expression level information of similar patient populations not known to have been treated with a Raf kinase inhibitor.
37. The method of 35, wherein baseline is the gene expression level of a reference standard.
38. The method of 37, wherein the reference standard is not obtained from the patient.
39. The method of 27, wherein the biological sample is obtained from lung, pancreas, thyroid, ovary, bladder, breast, prostate, liver, colon, myeloid tissue, skin, or tumor tissue.
40. The method of 37, wherein the reference standard is a reference sample of the same tissue type as the biological sample obtained from the patient.
41. The method of 27, wherein the biological sample is from a region showing evidence of a cell proliferative disorder.
42. The method of 35, wherein the biomarker is over-expressed in the biological sample relative to baseline.
43. The method of 35, wherein the biomarker is under-expressed in the biological sample relative to baseline.
44. The method of 31 further comprising administering a different Raf kinase inhibitor to the patient after the evaluating step.
45. The method of 28 further comprising adjusting the dosage amount for subsequent administration of the same or a different Raf kinase inhibitor to the patient.
46. The method of 27 or 35, wherein the determining step further comprises determining presence of gene expression of at least one biomarker selected from a subset of Table I represented by any of Tables II through VII.
47. The method of 27 or 35, wherein the determining step further comprises determining presence of gene expression of at least one biomarker selected from a subset of Table VIII represented by any of Tables IX through XX.
48. The method of 27 or 35, wherein the determining step further comprises determining presence of gene expression of at least two biomarkers selected from Table I and/or Table VIII.
49. The method of 27 or 35, wherein the determining step comprises detecting RNA transcribed by the at least one biomarker.
50. The method of 27 or 35, wherein the determining step comprises detecting DNA produced from reverse transcription of an RNA transcribed by the at least one biomarker.
51. The method of 27 or 35, wherein the determining step comprises detecting a polypeptide or protein encoded by the at least one biomarker.
52. The method of 27 or 35, wherein the at least one biomarker is operably linked to a gene chip.
53. The method of 27 or 35, wherein the at least one biomarker is represented in computer readable format.
54. The method of 27 or 35, wherein the determining step comprises contacting the biological sample with a gene chip comprising any of the biomarkers of Table I and/or Table VIII.
55. The method of 27, wherein the inhibitor is selected from the group consisting of CHIR-265; BAY 43-9006; ISIS-5132; CGP-69846A; ODN-698; ISIS-13650; LE-AON; LEraf-AON; LE-AON c-Raf; LE-5132; N-[3-[6-(3-Oxo-1,3-dihydro-2-benzofuran-5-ylamino)pyrazin-2-yl]phenyl]acetamide; PLX-4720; PLX-3204; PLX-3331; PLX-4718; PLX-4735; PLX-4032; N-[5-[3-(1-Cyano-1-methylethyl)benzamido]-2-methylphenyl]-4-oxo-3,4-dihydroquinazoline-6-carboxamide; and N-[5-[3-(1-Cyano-1-methylethyl)benzamido]-2-methylphenyl]-3-methyl-4-oxo-3,4-dihydroquinazoline-6-carboxamide.
56. The method of 55, wherein the inhibitor is CHIR-265.
57. The method of 27, wherein the biomarker is HGF.
58. The method of 27, wherein the biomarker is not HGF.
59. A method of treating a cell proliferative disorder, the method comprising

selecting a patient evidencing gene expression of a first set of at least one biomarker selected from Table I or Table VIII, and

administering to the patient a therapeutically effective amount of an agent that alters the level of gene expression compared to baseline of a second set of at least one biomarker selected from Table I or Table VIII.

60. The method of 59, wherein the first set and the second set include at least one of the same biomarker.
61. The method of 59, wherein the first set and the second set include the same biomarkers.
62. The method of 59, wherein the first set and/or the second set are selected from a subset of Table I represented by any of Tables II through VII.
63. The method of 59, wherein the first set and/or the second set are selected from a subset of Table VIII represented by any of Tables IX through XX.
64. The method of 59, wherein evidencing gene expression comprises determining presence of gene expression in a first biological sample obtained from the patient by detection of any of RNA transcribed by the first set, DNA produced from reverse transcription of an RNA transcribed by the first set, or a polypeptide or protein encoded by the first set.
65. The method of 64, wherein the alteration of gene expression compared to baseline is determined by measuring in a second biological sample obtained from the patient the quantity of any of RNA transcribed by the second set, DNA produced from reverse transcription of an RNA transcribed by the second set, or a polypeptide or protein encoded by the second set, and comparing the measured gene expression level to baseline.
66. The method of 65, wherein baseline is obtained from gene expression level information of similar patient populations not known to have been treated with a Raf kinase inhibitor.
67. The method of 65, wherein baseline is the gene expression level of a reference standard.
68. The method of 67, wherein the reference standard is not obtained from the patient.
69. The method of 64 or 65, wherein the biological sample is obtained from lung, pancreas, thyroid, ovary, bladder, breast, prostate, liver, colon, myeloid tissue, skin, or tumor tissue.
70. The method of 67, wherein the reference standard is a reference sample of the same tissue type as the second biological sample obtained from the patient.
71. The method of 65, wherein the second biological sample is from a region showing evidence of a cell proliferative disorder.
72. The method of 59, wherein the cell proliferative disorder is a neoplastic disorder.
73. The method of 72, wherein the neoplastic disorder is a cancer of any of lung, pancreas, thyroid, ovary, bladder, breast, prostate, liver, colon, myeloid tissue, or skin.
74. The method of claim 73, wherein the cancer is melanoma.
75. The method of claim 75, wherein the melanoma exhibits a V600E B-Raf mutation.
76. The method of 59, wherein the first set and/or the second set are operably linked to at least one gene chip.
77. The method of 59, wherein the inhibitor is selected from the group consisting of CHIR-265; BAY 43-9006; ISIS-5132; CGP-69846A; ODN-698; ISIS-13650; LE-AON; LEraf-AON; LE-AON c-Raf; LE-5132; N-[3-[6-(3-Oxo-1,3-dihydro-2-benzofuran-5-ylamino)pyrazin-2-yl]phenyl]acetamide; PLX-4720; PLX-3204; PLX-3331; PLX-4718; PLX-4735; PLX-4032; N-[5-[3-(1-Cyano-1-methylethyl)benzamido]-2-methylphenyl]-4-oxo-3,4-dihydroquinazoline-6-carboxamide; and N-[5-[3-(1-Cyano-1-methylethyl)benzamido]-2-methylphenyl]-3-methyl-4-oxo-3,4-dihydroquinazoline-6-carboxamide.
78. The method of 77, wherein the inhibitor is CHIR-265.
79. A method of identifying an agent for treatment of a cell proliferative disorder, the method comprising:

contacting the agent with a cell line or tissue associated with the disorder, and

testing a portion of the cell culture or tissue after the contacting to measure gene expression level of at least one biomarker selected from Table I or Table VIII that has been altered compared to baseline,

wherein detection of an alteration in expression level of the at least one biomarker is indicative of an identification of the agent for the treatment.

80. The method of 79, wherein the agent is a Raf kinase inhibitor.
81. The method of 79, wherein the at least one biomarker is selected from a subset of Table I represented by any of Tables II through VII.
82. The method of 79, wherein the at least one biomarker is selected from a subset of Table VIII represented by any of Tables IX through XX.
83. The method of 79, wherein the measurement of gene expression compared to baseline is measured by detecting the quantity of any of RNA transcribed by the at least one biomarker, DNA produced from reverse transcription of an RNA transcribed by the at least one biomarker, or a polypeptide or protein encoded by the at least one biomarker.
84. The method of 79, wherein baseline is obtained from gene expression level information of similar patient populations not known to have been treated with a Raf kinase inhibitor.
85. The method of 79, wherein baseline is the gene expression level of a reference standard.
86. The method of 79, wherein the at least one biomarker is operably linked to a gene chip.
87. A method of identifying a Raf kinase inhibitory agent for treatment of a cell proliferative disorder or to guide a decision to progress a Raf kinase inhibitory agent to further development, the method comprising:

contacting the agent with A375M cells or cells from an A375M-cell-initiated xenograft tumor, and

testing a portion of the contacted cells to measure gene expression level of at least one biomarker selected from Table I or Table VIII,

wherein detection of an alteration in expression level compared to baseline of the at least one biomarker is indicative of an identification of the agent for the treatment or a favorable decision to progress the compound for further development.

88. The method of 87, wherein the at least one biomarker is selected from a subset of Table I represented by any of Tables II through VII.
89. The method of 87, wherein the at least one biomarker is selected from a subset of Table VIII represented by any of Tables IX through XX.
90. The method of 87, wherein the measurement of gene expression is measured by detecting the quantity of any of RNA transcribed by the at least one biomarker, DNA produced from reverse transcription of an RNA transcribed by the at least one biomarker, or a polypeptide or protein encoded by the at least one biomarker.
91. The method of 87, wherein the at least one biomarker is operably linked to a gene chip.
92. A data set presented in a computer readable format for indicating response to a Raf kinase inhibitor, the data set comprising the biomarkers of Table I and/or Table VIII.
93. The data set of 92, wherein the data set comprises a subset of Table I represented by any of Tables II through VII.
94. The data set of 92, wherein the data set comprises a subset of Table VIII represented by any of Tables IX through XX.
95. The data set of 93 or 94, wherein the biomarkers are operably linked to a gene chip. All references presented herein are incorporated by reference in their entirety as if fully set forth herein.

LENGTHY TABLES
The patent application contains a lengthy table section. A copy of the table is available in electronic form from the USPTO web site (http://seqdata.uspto.gov/?pageRequest=docDetail&DocID=US20100004253A1). An electronic copy of the table will also be available from the USPTO upon request and payment of the fee set forth in 37 CFR 1.19(b)(3).

1. A method of identifying a patient for treatment with a Raf kinase inhibitor, the method comprising:
determining presence of gene expression of at least one biomarker selected from Table I or Table VIII in a biological sample obtained from the patient,
identifying the patient for treatment if the presence of gene expression of the at least one biomarker is detected.

2. The method of claim 1, wherein the determining step further comprises measuring gene expression level of the at least one biomarker and comparing the measured gene expression level to baseline.

3. The method of claim 1, wherein the biological sample is obtained from lung, pancreas, thyroid, ovary, bladder, breast, prostate, liver, colon, myeloid tissue, skin, or tumor tissue.

4. The method of claim 1, wherein the biological sample is from a region showing evidence of a cell proliferative disorder.

5. The method of claim 2, wherein the biomarker is over-expressed in the biological sample relative to baseline.

6. The method of claim 2, wherein the biomarker is under-expressed in the biological sample relative to baseline.

7. The method of claim 1, wherein the determining step further comprises determining gene expression of at least one biomarker selected from a subset of Table I represented by any of Tables II through VII.

8. The method of claim 1, wherein the determining step further comprises determining gene expression of at least one biomarker selected from a subset of Table VIII represented by any of Tables IX through XX.

9. The method of claim 1, wherein the determining step further comprises determining gene expression of at least two biomarkers selected from Table I and/or Table VIII.

10. The method of claim 1, wherein the inhibitor is selected from the group consisting of CHIR-265; BAY 43-9006; ISIS-5132; CGP-69846A; ODN-698; ISIS-13650; LE-AON; LEraf-AON; LE-AON c-Raf; LE-5132; N-[3-[6-(3-Oxo-1,3-dihydro-2-benzofuran-5-ylamino)pyrazin-2-yl]phenyl]acetamide; PLX-4720; PLX-3204; PLX-3331; PLX-4718; PLX-4735; PLX-4032; N-[5-[3-(1-Cyano-1-methylethyl)benzamido]-2-methylphenyl]-4-oxo-3,4-dihydroquinazoline-6-carboxamide; and N-[5-[3-(1-Cyano-1-methylethyl)benzamido]-2-methylphenyl]-3-methyl-4-oxo-3,4-dihydroquinazoline-6-carboxamide.

11. A method of monitoring response of a patient to treatment with a Raf kinase inhibitor, the method comprising:
determining presence of gene expression of at least one biomarker selected from Table I or Table VIII in a biological sample obtained from a patient who has been administered a Raf kinase inhibitor,
evaluating response of the patient based on detection of the presence of gene expression of the at least one biomarker.

12. The method of claim 11, wherein the inhibitor has been administered in a therapeutically effective amount.

13. The method of claim 11 further comprising the step of administering an amount of a Raf kinase inhibitor to the patient.

14. The method of claim 13, wherein the inhibitor is administered before the biological sample is obtained from the patient.

15. The method of claim 13, wherein the inhibitor is administered after the evaluating step.

16. The method of claim 11 further comprising obtaining a biological sample from the patient subsequent to the administration of the Raf kinase inhibitor.

17. The method of claim 11, wherein the determining step further comprises measuring gene expression level of the at least one biomarker and comparing the measured gene expression level to baseline.

18. The method of claim 15 further comprising administering a different Raf kinase inhibitor to the patient after the evaluating step.

19. The method of claim 12 further comprising adjusting the dosage amount for subsequent administration of the same or a different Raf kinase inhibitor to the patient.

20. A method of treating a cell proliferative disorder, the method comprising
selecting a patient evidencing gene expression of a first set of at least one biomarker selected from Table I or Table VIII, and
administering to the patient a therapeutically effective amount of an agent that alters the level of gene expression compared to baseline of a second set of at least one biomarker selected from Table I or Table VIII.

21. The method of claim 20, wherein the first set and the second set include at least one of the same biomarker.

22. The method of claim 20, wherein the first set and the second set include the same biomarkers.

23. The method of claim 20, wherein the cell proliferative disorder is a neoplastic disorder.

24. The method of claim 23, wherein the neoplastic disorder is a cancer of any of lung, pancreas, thyroid, ovary, bladder, breast, prostate, liver, colon, myeloid tissue, or skin.

25. The method of claim 24, wherein the cancer is melanoma.

26. The method of claim 25, wherein the melanoma exhibits a V600E B-Raf mutation.

27. The method of claim 20, wherein the first set and/or the second set are operably linked to at least one gene chip.

28. A method of identifying an agent for treatment of a cell proliferative disorder, the method comprising:
contacting the agent with a cell line or tissue associated with the disorder, and
testing a portion of the cell culture or tissue after the contacting to measure gene expression level of at least one biomarker selected from Table I or Table VIII that has been altered compared to baseline,
wherein detection of an alteration in expression level of the at least one biomarker is indicative of an identification of the agent for the treatment.

29. The method of claim 28, wherein the agent is a Raf kinase inhibitor.

30. The method of claim 28, wherein the measurement of gene expression compared to baseline is measured by detecting the quantity of any of RNA transcribed by the at least one biomarker, DNA produced from reverse transcription of an RNA transcribed by the at least one biomarker, or a polypeptide or protein encoded by the at least one biomarker.
